(19) United States
(12) Patent Application Publication
(10) Pub. No.: US 2025/0249012 A1
(43) Pub. Date: Aug. 7, 2025

(54) COMPOSITIONS AND METHODS FOR TREATING AND/OR PREVENTING A VIRAL INFECTION

(71) Applicants: The Children's Medical Center Corporation, Boston, MA (US); University of Helsinki, Helsinki (FI)

(72) Inventors: Tomas Kirchhausen, Brighton, MA (US); Alex Kreutzberger, Boston, MA (US); Giuseppe Balistreri, Helsinki (FI)

(73) Assignees: The Children's Medical Center Corporation, Boston, MA (US); University of Helsinki, Helsinki (FI)

(21) Appl. No.: 18/854,929

(22) PCT Filed: Apr. 7, 2023
(86) PCT No.: PCT/US2023/017858

§ 371 Date: Oct. 7, 2024

Related U.S. Application Data
(60) Provisional application No. 63/329,235, filed on Apr. 8, 2022.

(51) Int. Cl. A61K 31/5377; A61K 9/00; A61K 9/08; A61K 31/245; A61K 47/10; A61K 47/20; A61K 47/26; A61P 31/14

(52) U.S. Cl. CPC A61K 31/5377; A61K 9/0043; A61K 9/08; A61K 31/245; A61K 47/10; A61K 47/20; A61K 47/26; A61P 31/14

(57) ABSTRACT

The present disclosure provides compositions comprising a phosphatidylinositol-3-phosphate 5-kinase (PIKfyve kinase) inhibitor, or a pharmaceutically acceptable salt, solvate, hydrate, stereoisomer, polymorph, tautomer, isotopically enriched form, or prodrug thereof; a serine protease inhibitor, or a pharmaceutically acceptable salt, solvate, hydrate, stereoisomer, polymorph, tautomer, isotopically enriched form, or prodrug thereof; and water. Also provided in the present disclosure are methods, uses, and kits involving the compositions for treating and/or preventing a viral infection (e.g., infection by a coronavirus, an ebolavirus, a Lassa virus) in a subject. Also provided are methods, uses, and kits involving the compositions for treating and/or preventing damage to respiratory tissue (e.g. upper respiratory tissue, for example, nasal tissue, nasopharyngeal tissue, and/or lower respiratory tissue, for example, lung tissue) in a subject.

### GOVERNMENT SUPPORT

This invention was made with government support under Grant Nos. GM130386 and AI163019, awarded by the National Institutes of Health. The Government has certain rights in the invention.

### BACKGROUND

The coronavirus SARS-CoV-2 is the causative agent of the COVID-19 pandemic. Prevention and/or minimization of infection by SARS-CoV-2 and related coronaviruses decreases the risk of developing COVID-19, SARS, or other diseases associated with coronavirus infection, and can be achieved by simultaneous targeting of host entry factors. These factors include proteins, lipids and related molecules associated and/or required to regulate pathways of intracellular vesicular transport and/or membrane traffic and the trafficking, sorting and availability of host factors that modulate properties of the invading virus required for cell entry.

Apilimod, Camostat, and Nafamostat are drugs in clinical trial to explore efficacy for potential treatment of COVID-19, and they are administered orally and intravenously (IV), respectively. They are well tolerated for periods spanning days, weeks and even years. Their availability in respiratory tissues is unknown. Oral administration of Apilimod has entered a Phase I clinical trial to treat COVID-19 patients. Camostat has been tested with hospitalized COVID-19 patients in Denmark, and is in clinical trials on adult COVID-19 patients in France. Nafamostat is in COVID-19 related clinical trials in Russia, South Korea, and Japan.

In particular, it would be important to develop a novel approach to inhibiting SARS-CoV-2 infection and preventing damage of respiratory tissue, such as lung tissue damage, due to this infection.

### SUMMARY

It has been shown that SARS-CoV-2 entry to the host involves a pH dependent fusion step; fusion is inactive at neutral pH, and depending on the variants, becomes optimal ˜pH 6.5-6.8. In general, this fusion occurs in endosomal compartments and requires virus uptake; fusion can also occur at the plasma membrane, if pH of the cell environment is slightly acidic (pH ˜6.6-6.8). Surface S glycoprotein of SARS-CoV-2 proteolysis mediated by Furin/TMPRSS2 or Furin/Cathepsin allows for fusion between the membranes of the virus and host. Inhibition of TMPRSS2 (and related serine proteases) or PIKfyve kinase prevents fusion, by virtue of directly inhibiting serine protease activity (Nafamostat and Camostat), or indirectly preventing fusion mediated by cathepsin activity (Apilimod and related compounds including Vacuolin family members), respectively.

A surprising and unexpected potent synergy of the combined use of inhibitors of serine proteases and PIKfyve kinase was observed. It was surprising because the targets of the inhibitors are unique and different proteins located in different cellular environments. The observed synergy was also present even if the host cell only had intrinsic TMPRSS2 but not Cathepsin activity. This opened the possibility of combining drugs effective for both targets to strongly prevent virus infection even in cells with low or no cathepsin activity, as known to occur in many cell types along the respiratory tract.

Because the first tissue targeted for SARS-CoV-2 infection maps to the nasopharyngeal cavity, it was reasoned that direct administration of inhibitors to this tissue could provide a more effective way to reach the cells infected by the virus; it would allow use of lower concentrations and vastly smaller amounts of drugs, significantly minimize solubility issues for the drugs due to the low amounts for local delivery required, would facilitate drug formulation, and would enhance the bioactivity of the drugs in the nasopharyngeal cavity while minimizing distribution to other tissues in the body and at the same time vastly diluting the concentration of inhibitors should they reach such other tissues.

Mice exposed to human isolates of the authentic SARS-CoV-2 beta variant (this variant is efficiently recognized by the mouse ACE2 receptor, and hence infects non-transgenic mice) show: (1) Complete inhibition of SARS-CoV-2 infection in lungs of mice exposed first to a water solution containing Apilimod dimesylate and Nafamostat mesylate and 1 min later to virus by intranasal instillation. Substantial reduction of at least 220-fold to undetectable levels in infection compared to control untreated mice, 2 days after virus exposure demonstrated by PCR from RNA samples extracted from lower respiratory tissues (lung); absence of detectable infected cells and complete absence of dead cells determined by pathology (specific staining for cell infection and cell death). Same results were obtained using decreasing doses and different combinations of Nafamostat mesylate and Apilimod dimesylate (4+1, 4+2, 0.8+0.2, 0.4+0.2 mg/Kg); this dosing compares favorably to 600-1200 mg/kg Pfizer Paxlovid administered orally in mice. (2) Complete inhibition of cell death and lung tissue damage induced by SARS-CoV-2 infection on mice exposed first to virus by intranasal instillation followed 6 hours later by intranasal administration of a water solution containing Apilimod and Nafamostat. Equivalent observations as in #1 were done using 0.8+0.2 mg/Kg of Apilimod dimesylate/Nafamostat mesylate.

Combined intranasal administration in mice using very low amounts of Apilimod mesylate and Nafamostat mesylate dissolved in water strongly blocks SARS-CoV-2 infection and lung tissue damage due to infection. This contrasts with the lack of antiviral effect upon sole administration of Apilimod dimesylate (oral, IP or intranasal), or the partial antiviral inhibitory effect obtained by treatments with Nafamostat mesylate (IV or intranasal); in these cases, the single drugs are ineffective or partially effective to treat and/or prevent SARS-CoV-2 infection in animals or humans.

Full inhibition of infection follows intranasal delivery of combined mixture of Apilimod dimesylate and Nafamostat mesylate and virus. Potent block of infection was determined by PCR, and there were no signs of tissue damage (apoptosis) in mice evaluated 2 days post infection if drugs and virus are administered within a minute of each other. No block of infection was observed by PCR, but there were no signs of tissue damage (apoptosis) in mice evaluated 2 days post infection if drugs are administered 6 hours post virus exposure.

The pharmacokinetics in plasma of Apilimod dimesylate and Nafamostat mesylate indicate relatively short half-life times (minutes). This is advantageous when considering intranasal delivery to efficiently reach the target nasopharyngeal tissues while at the same time clearing the drugs that might reach the blood stream thereby minimizing potential side effects.

This disclosure encompasses the intranasal administration by single, or multiple combined applications of PIKfyve kinase inhibitors and serine protease inhibitors to individuals before exposure to SARS-CoV-2 or during early stages of infection. The PIKfyve kinase inhibitors include Apilimod dimesylate and related compounds including Vacuolin family members; the serine protease inhibitors include Nafamostat mesylate and Camostat mesylate. The discovery takes advantage of the following properties: (1) demonstration of potent synergy of inhibition by the combined use of the inhibitors targeting PIKfyve and serine proteases including TMPRSS2 (Kreutzberger et al JV, 2021); (2) intranasal nasal delivery of an aqueous solution (to increase drug solubility) of Apilimod dimesylate and Nafamostat mesylate allows rapid, efficient, and direct delivery to target cells and tissues within the nasopharyngeal cavity, and vastly reduces the amounts and concentrations of drugs required for active biological action.

In one aspect, the present disclosure provides compositions (e.g. pharmaceutical compositions) comprising:
(a) a phosphatidylinositol-3-phosphate 5-kinase (PIKfyve kinase) inhibitor, or a pharmaceutically acceptable salt, solvate, hydrate, stereoisomer, polymorph, tautomer, isotopically enriched form, or prodrug thereof;
(b) a serine protease inhibitor, or a pharmaceutically acceptable salt, solvate, hydrate, stereoisomer, polymorph, tautomer, isotopically enriched form, or prodrug thereof, and
(c) water.

In some embodiments, the composition (e.g. pharmaceutical composition) comprises a solution of:
(a) a phosphatidylinositol-3-phosphate 5-kinase (PIKfyve kinase) inhibitor, or a pharmaceutically acceptable salt, solvate, hydrate, stereoisomer, polymorph, tautomer, isotopically enriched form, or prodrug thereof;
(b) a serine protease inhibitor, or a pharmaceutically acceptable salt, solvate, hydrate, stereoisomer, polymorph, tautomer, isotopically enriched form, or prodrug thereof, and
(c) water.

In certain embodiments, a composition described herein includes a therapeutically and/or prophylactically effective amount of a compound described herein. In certain embodiments, the composition is a pharmaceutical composition. The compositions may be useful in treating and/or preventing a viral infection (e.g., infection by a coronavirus, an ebolavirus, a Lassa virus) in a subject. The compositions may be useful in treating and/or preventing damage to respiratory tissue (e.g. upper respiratory tissue, for example, nasal tissue, nasopharyngeal tissue, and/or lower respiratory tissue, for example, lung tissue) in a subject.

In certain embodiments, the composition includes apilimod (e.g. apilimod dimesylate). In certain embodiments, the composition includes apilimod dimesylate. In certain embodiments, the composition includes nafamostat (e.g. nafamostat mesylate). In certain embodiments, the composition includes nafamostat mesylate. In certain embodiments, the composition includes apilimod (e.g. apilimod dimesylate) and nafamostat (e.g. nafamostat mesylate). In certain embodiments, the composition includes apilimod dimesylate and nafamostat mesylate.

In certain embodiments, the composition comprises about 1-50 mg/mL or about 50-100 mg/mL apilimod dimesylate. In certain embodiments, the composition comprises about 1-25 mg/mL, about 25-50 mg/mL, about 50-75 mg/mL, or about 75-100 mg/mL apilimod dimesylate. In certain embodiments, the composition comprises about 1-20 mg/mL, about 20-40 mg/mL, about 40-60 mg/mL, about 60-80 mg/mL, or about 80-100 mg/mL apilimod dimesylate. In certain embodiments, the composition comprises about 1-10 mg/mL, about 10-20 mg/mL, about 20-30 mg/mL, about 30-40 mg/mL, about 40-50 mg/mL, about 50-60 mg/mL, about 60-70 mg/mL, about 70-80 mg/mL, about 80-90 mg/mL, or about 90-100 mg/mL apilimod dimesylate. In certain embodiments, the composition comprises about 10-30 mg/mL apilimod dimesylate. In certain embodiments, the composition comprises about 15-20 mg/mL (e.g. about 18 mg/mL) apilimod dimesylate. In certain embodiments, the composition comprises about 18 mg/ml apilimod dimesylate.

In certain embodiments, the composition comprises about 1-100 mg/mL or about 100-200 mg/mL nafamostat mesylate. In certain embodiments, the composition comprises about 1-50 mg/mL, about 50-100 mg/mL, about 100-150 mg/mL, or about 150-200 mg/mL nafamostat mesylate. In certain embodiments, the composition comprises about 1-25 mg/mL, about 25-50 mg/mL, about 50-75 mg/mL, about 75-100 mg/mL, about 100-125 mg/mL, about 125-150 mg/mL, about 150-175 mg/mL, or about 175-200 mg/mL nafamostat mesylate. In certain embodiments, the composition comprises about 1-20 mg/mL, about 20-40 mg/mL, about 40-60 mg/mL, about 60-80 mg/mL, about 80-100 mg/mL, about 100-120 mg/mL, about 120-140 mg/mL, about 140-160 mg/mL, about 160-180 mg/mL, or about 180-200 mg/mL nafamostat mesylate. In certain embodiments, the composition comprises about 1-10 mg/mL, about 10-20 mg/mL, about 20-30 mg/mL, about 30-40 mg/mL, about 40-50 mg/mL, about 50-60 mg/mL, about 60-70 mg/mL, about 70-80 mg/mL, about 80-90 mg/mL, about 90-100 mg/mL, about 100-110 mg/mL, about 110-120 mg/mL, about 120-130 mg/mL, about 130-140 mg/mL, about 140-150 mg/mL, about 150-160 mg/mL, about 160-170 mg/mL, about 170-180 mg/mL, about 180-190 mg/mL, or about 190-200 mg/mL nafamostat mesylate. In certain embodiments, the composition comprises about 20-60 mg/mL nafamostat mesylate. In certain embodiments, the composition comprises about 30-40 mg/mL (e.g. about 36 mg/mL) nafamostat mesylate. In certain embodiments, the composition comprises about 36 mg/mL nafamostat mesylate.

In certain embodiments, the composition comprises about 1-100 mg/mL apilimod dimesylate (e.g. about 1-25 mg/mL, about 25-50 mg/mL, about 50-75 mg/mL or about 75-100 mg/mL) and about 1-200 mg/mL nafamostat mesylate (e.g. about 1-50 mg/mL, about 50-100 mg/mL, about 100-150 mg/mL, or about 150-200 mg/mL). In certain embodiments, the composition comprises about 1-100 mg/mL apilimod dimesylate (e.g. about 1-20 mg/mL, about 20-40 mg/mL, about 40-60 mg/mL, about 60-80 mg/mL, or about 80-100 mg/mL) and about 1-200 mg/mL nafamostat mesylate (e.g. about 1-40 mg/mL, about 40-80 mg/mL, about 80-120 mg/mL, about 120-160 mg/mL, or about 160-200 mg/mL). In certain embodiments, the composition comprises about 1-100 mg/mL apilimod dimesylate (e.g. about 1-10 mg/mL, about 10-20 mg/mL, about 20-30 mg/mL, about 30-40 mg/mL, about 40-50 mg/mL, about 50-60 mg/mL, about 60-70 mg/mL, about 70-80 mg/mL, about 80-90 mg/mL, or about 90-100 mg/mL) and about 1-200 mg/mL nafamostat mesylate (e.g. about 1-20 mg/mL, about 20-40 mg/mL, about 40-60 mg/mL, about 60-80 mg/mL, about 80-100 mg/mL, about 100-120 mg/mL, about 120-140 mg/mL, about 140-160 mg/mL, about 160-180 mg/mL, or about 180-200 mg/mL). In certain embodiments, the composition comprises about 10-30 mg/mL apilimod dimesylate and about 20-60 mg/mL nafamostat mesylate. In certain embodiments, the composition comprises about 15-20 mg/mL (e.g. about 18 mg/mL) apilimod dimesylate and about 30-40 mg/mL (e.g. about 36 mg/mL) nafamostat mesylate. In certain embodiments, the composition comprises about 18 mg/mL apilimod dimesylate and about 36 mg/mL nafamostat mesylate.

In another aspect, described herein are methods for treating and/or preventing a viral infection (e.g., infection by a coronavirus, an ebolavirus, a Lassa virus) in a subject, the methods comprising intranasal administration of a composition described herein. In certain embodiments, the virus is severe acute respiratory syndrome coronavirus 1 (SARS-CoV-1). In certain embodiments, the virus is severe acute respiratory syndrome coronavirus 2 (SARS-CoV-2). In certain embodiments, the virus is Middle East respiratory syndrome-related coronavirus (MERS-CoV). In certain embodiments, the virus causes coronavirus disease 2019 (COVID-19). In certain embodiments, the intranasal administration to the subject is before exposure of the subject to the virus. In certain embodiments, the intranasal administration to the subject is after exposure of the subject to the virus. In certain embodiments, the intranasal administration is via nasal drops. In certain embodiments, the intranasal administration is via a nasal spray. In certain embodiments, the intranasal administration is via inhalation, nebulization, or aerosolization. In certain embodiments, the intranasal administration comprises inhaling through the nose. In certain embodiments, the intranasal administration comprises inhaling through the mouth. In certain embodiments, the intranasal administration comprises inhaling through the nose and mouth.

In another aspect, described herein are methods for treating and/or preventing damage to respiratory tissue (e.g. upper respiratory tissue, for example, nasal tissue, nasopharyngeal tissue, and/or lower respiratory tissue, for example, lung tissue) in a subject, the methods comprising intranasal administration of a composition described herein. In certain embodiments, the respiratory tissue is upper respiratory tissue (e.g. nasal tissue, nasopharyngeal tissue). In certain embodiments, the upper respiratory tissue is nasal tissue or nasopharyngeal tissue. In certain embodiments, the respiratory tissue is lower respiratory tissue (e.g. lung tissue). In certain embodiments, the lower respiratory tissue is lung tissue. In certain embodiments, the intranasal administration to the subject is before exposure of the subject to the virus. In certain embodiments, the intranasal administration to the subject is after exposure of the subject to the virus. In certain embodiments, the intranasal administration is via nasal drops. In certain embodiments, the intranasal administration is via a nasal spray. In certain embodiments, the intranasal administration is via inhalation, nebulization, or aerosolization. In certain embodiments, the intranasal administration comprises inhaling through the nose. In certain embodiments, the intranasal administration comprises inhaling through the mouth. In certain embodiments, the intranasal administration comprises inhaling through the nose and mouth.

In another aspect, described herein are uses of a composition described herein to treat and/or prevent a viral infection (e.g., infection by a coronavirus, an ebolavirus, a Lassa virus) caused by a virus in a subject in need thereof. In certain embodiments, the virus is severe acute respiratory syndrome coronavirus 1 (SARS-CoV-1). In certain embodiments, the virus is severe acute respiratory syndrome coronavirus 2 (SARS-CoV-2). In certain embodiments, the virus is Middle East respiratory syndrome-related coronavirus (MERS-CoV). In certain embodiments, the virus causes coronavirus disease 2019 (COVID-19).

In another aspect, described herein are uses of a composition described herein to treat and/or prevent damage to respiratory tissue (e.g. upper respiratory tissue, for example, nasal tissue, nasopharyngeal tissue, and/or lower respiratory tissue, for example, lung tissue) in a subject in need thereof. In certain embodiments, the respiratory tissue is upper respiratory tissue (e.g. nasal tissue, nasopharyngeal tissue). In certain embodiments, the upper respiratory tissue is nasal tissue or nasopharyngeal tissue. In certain embodiments, the respiratory tissue is lower respiratory tissue (e.g. lung tissue). In certain embodiments, the lower respiratory tissue is lung tissue.

In another aspect, described herein are kits comprising a container with a compound, or pharmaceutical composition thereof, as described herein. The kits described herein may include a single dose or multiple doses of the compound or pharmaceutical composition. The kits may be useful in a method of the disclosure. In certain embodiments, the kit further includes instructions for using the compound or pharmaceutical composition. A kit described herein may also include information (e.g. prescribing information) as required by a regulatory agency, such as the U.S. Food and Drug Administration (FDA).

The details of one or more embodiments of the disclosure are set forth herein. Other features, objects, and advantages of the disclosure will be apparent from the Detailed Description, Examples, Figures, and Claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1B show the solubility of Apilimod and Nafamostat in different vehicles. FIG. 1A shows the drugs solubilized at 1 mg/ml (Apilimod di-mesylate) and 2 mg/ml (Nafamostat mesylate) in either Dulbecco's Modified Eagle Medium (DMEM), phosphate buffered saline (PBS), or deionized water (d-H₂O). After incubation for 12 hours at room temperature, a precipitate is observable in the solutions prepared in DMEM and PBS, but not in d-H₂O. FIG. 1B shows that after centrifugation at 10.000×g for 1 minute, room temperature, the precipitate is visible at the bottom of the tubes containing DMEM and PBS (arrows) but not in the tubes where the drugs were solubilized in d-H₂O. Magnified images of the boxed areas are shown on the right panels.

FIGS. 2A-2D show antiviral activity and cell toxicity of Apilimod and Nafamostat prepared in different vehicles. FIGS. 2A-2B show antiviral activity of apilimod (FIG. 2A) and nafamostat (FIG. 2B) prepared at 1 mg/ml (apilimod) and 2 mg/ml (nafamostat) in either deionized water (d-water), phosphate buffered saline (PBS), or DMEM medium (DMEM). 30 min prior to infection with SARS-CoV-2 Wuhan strain, Vero-E6 cells (apilimod) and A549-ACE2-TMPRSS2GFP cells (Nafamostat) were pre-treated with apilimod or nafamostat dissolved in the indicated vehicles and subsequently diluted at the indicated concentrations in DMEM containing 2% FBS. Cells were fixed at 20 hours post infection (hpi) and the number of infected cells determined after immunofluorescence localization of the viral protein N and cell nuclei using Hoechst DNA dye followed by high-content imaging and automated image analysis. FIGS. 2C-2D show the number of cells in each experiment determined after fluorescence imaging by automatic detection of Hoechst-stained nuclei. FIG. 2C shows the number of cells after treatment with apilimod as described above. FIG. 2D shows the number of cells after treatment with nafamostat as described above. Values are normalized to the number of cells obtained in parallel experiments where infected cells were treated only with the respective vehicles. Significant loss of cells was observed in experiments where the drugs stocks had been prepared in DMEM and PBS, but not when prepared in deionized water. All values are the mean and STDEV of three independent experiments.

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

When a range of values (“range”) is listed, it encompasses each value and sub-range within the range. A range is inclusive of the values at the two ends of the range unless otherwise provided. For example, the range from 1 to 4 includes 1 and 4.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO_{3″}⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline carth metal, ammonium and N⁺(C₁₋₄ alkyl)₄ salts. Representative alkali or alkaline carth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline carth metal, ammonium, and N⁺(C₁₋₄ alkyl)₄ salts. Representative alkali or alkaline carth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or basc. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stercoisomers”.

Stercoisomers that are not mirror images of one another are termed “diastercomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “amorphous” or “amorphous form” refers to a form of a solid (“solid form”), the form substantially lacking three-dimensional order. In certain embodiments, an amorphous form of a solid is a solid form that is substantially not crystalline. In certain embodiments, the X-ray powder diffraction (XRPD) pattern of an amorphous form includes a wide scattering band with a peak at 2θ of, e.g., between 20 and 70°, inclusive, using CuKα radiation. In certain embodiments, the XRPD pattern of an amorphous form further includes one or more peaks attributed to crystalline structures. In certain embodiments, the maximum intensity of any one of the one or more peaks attributed to crystalline structures observed at a 2θ of between 20 and 70°, inclusive, is not more than 300-fold, not more than 100-fold, not more than 30-fold, not more than 10-fold, or not more than 3-fold of the maximum intensity of the wide scattering band. In certain embodiments, the XRPD pattern of an amorphous form includes no peaks attributed to crystalline structures.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed hercin. In certain embodiments, in the co-crystal, there is partial proton transfer from the acid to a compound disclosed herein. Co-crystals may be useful to improve the properties (e.g., solubility, stability, and case of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (scc, Bundgaard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C1-C8 alkyl, C2-C8 alkenyl, C2-C8 alkynyl, aryl, C7-C12 substituted aryl, and C7-C12 arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering.” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for inhibiting PIKfyve kinase. In certain embodiments, a therapeutically effective amount is an amount sufficient for inhibiting a serine protease (e.g. TMPRSS2). In certain embodiments, a therapeutically effective amount is an amount sufficient for treating a viral infection (e.g., infection by a coronavirus, an ebolavirus, a Lassa virus) in a subject. In certain embodiments, a therapeutically effective amount is an amount sufficient for inhibiting PIKfyve kinase and/or inhibiting a serine protease (e.g. TMPRSS2). In certain embodiments, a therapeutically effective amount is an amount sufficient for inhibiting PIKfyve kinase, inhibiting a serine protease (e.g. TMPRSS2), and/or treating a viral infection (e.g., infection by a coronavirus, an ebolavirus, a Lassa virus) in a subject.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting PIKfyve kinase. In certain embodiments, a prophylactically effective amount is an amount sufficient for inhibiting a serine protease (e.g. TMPRSS2). In certain embodiments, a prophylactically effective amount is an amount sufficient for preventing a viral infection (e.g., infection by a coronavirus, an ebolavirus, a Lassa virus) in a subject. In certain embodiments, a prophylactically effective amount is an amount sufficient for inhibiting PIKfyve kinase and/or inhibiting a serine protease (e.g. TMPRSS2). In certain embodiments, a prophylactically effective amount is an amount sufficient for inhibiting PIKfyve kinase, inhibiting a serine protease (e.g. TMPRSS2), and/or preventing a viral infection (e.g., infection by a coronavirus, an ebolavirus, a Lassa virus) in a subject.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

As used herein the term “inhibit” or “inhibition” in the context of enzymes, for example, in the context of PIKfyve kinase or a serine protease (e.g. TMPRSS2) refers to a reduction in the activity of the enzyme. In some embodiments, the term refers to a reduction of the level of enzyme activity, e.g., PIKfyve kinase or a serine protease (e.g. TMPRSS2) activity, to a level that is statistically significantly lower than an initial level, which may, for example, be a baseline level of enzyme activity. In some embodiments, the term refers to a reduction of the level of enzyme activity, e.g., PIKfyve kinase or a serine protease (e.g. TMPRSS2) activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

A “kinase” is a type of enzyme that transfers phosphate groups from high energy donor molecules, such as ATP, to specific substrates, referred to as phosphorylation. Kinases are part of the larger family of phosphotransferases. One of the largest groups of kinases are protein kinases, which act on and modify the activity of specific proteins. Kinases are used extensively to transmit signals and control complex processes in cells. Various other kinases act on small molecules such as lipids, carbohydrates, amino acids, and nucleotides, either for signaling or to prime them for metabolic pathways. Kinases are often named after their substrates. More than 500 different protein kinases have been identified in humans. These exemplary human protein kinases include, but are not limited to, AAK1, ABL, ACK, ACTR2, ACTR2B, AKT1, AKT2, AKT3, ALK, ALK1, ALK2, ALK4, ALK7, AMPKa1, AMPKa2, ANKRD3, ANPa, ANPb, ARAF, ARAFps, ARG, AurA, AurAps1, AurAps2, AurB, AurBps1, AurC, AXL, BARK1, BARK2, BIKE, BLK, BMPR1A, BMPR1Aps1, BMPR1Aps2, BMPR1B, BMPR2, BMX, BRAF, BRAFps, BRK, BRSK1, BRSK2, BTK, BUB1, BUBR1, CaMK1a, CaMK1b, CaMK1d, CaMK1g, CaMK2a, CaMK2b, CaMK2d, CaMK2g, CaMK4, CaMKK1, CaMKK2, caMLCK, CASK, CCK4, CCRK, CDC2, CDC7, CDK10, CDK11, CDK2, CDK3, CDK4, CDK4ps, CDK5, CDK5ps, CDK6, CDK7, CDK7ps, CDK8, CDK8ps, CDK9, CDKL1, CDKL2, CDKL3, CDKL4, CDKL5, CGDps, CHED, CHK1, CHK2, CHK2ps1, CHK2ps2, CK1a, CK1a2, CK1aps1, CK1aps2, CK1aps3, CK1d, CK1e, CK1g1, CK1g2, CK1g2ps, CK1g3, CK2a1, CK2a1-rs, CK2a2, CLIK1, CLIK1L, CLK1, CLK2, CLK2ps, CLK3, CLK3ps, CLK4, COT, CRIK, CRK7, CSK, CTK, CYGD, CYGF, DAPK1, DAPK2, DAPK3, DCAMKL1, DCAMKL2, DCAMKL3, DDR1, DDR2, DLK, DMPK1, DMPK2, DRAK1, DRAK2, DYRK1A, DYRK1B, DYRK2, DYRK3, DYRK4, EGFR, EphA1, EphA10, EphA2, EphA3, EphA4, EphA5, EphA6, EphA7, EphA8, EphB1, EphB2, EphB3, EphB4, EphB6, Erk1, Erk2, Erk3, Erk3ps1, Erk3ps2, Erk3ps3, Erk3ps4, Erk4, Erk5, Erk7, FAK, FER, FERps, FES, FGFR1, FGFR2, FGFR3, FGFR4, FGR, FLT1, FLT1ps, FLT3, FLT4, FMS, FRK, Fused, FYN, GAK, GCK, GCN2, GCN22, GPRK4, GPRK5, GPRK6, GPRK6ps, GPRK7, GSK3A, GSK3B, Haspin, HCK, HER2/ErbB2, HER3/ErbB3, HER4/ErbB4, HH498, HIPK1, HIPK2, HIPK3, HIPK4, HPK1, HRI, HRIps, HSER, HUNK, ICK, IGF1R, IKKa, IKKb, IKKe, ILK, INSR, IRAK1, IRAK2, IRAK3, IRAK4, IRE1, IRE2, IRR, ITK, JAK1, JAK2, JAK3, JNK1, JNK2, JNK3, KDR, KHS1, KHS2, KIS, KIT, KSGCps, KSR1, KSR2, LATS1, LATS2, LCK, LIMK1, LIMK2, LIMK2ps, LKB1, LMR1, LMR2, LMR3, LOK, LRRK1, LRRK2, LTK, LYN, LZK, MAK, MAP2K1, MAP2K1ps, MAP2K2, MAP2K2ps, MAP2K3, MAP2K4, MAP2K5, MAP2K6, MAP2K7, MAP3K1, MAP3K2, MAP3K3, MAP3K4, MAP3K5, MAP3K6, MAP3K7, MAP3K8, MAPKAPK2, MAPKAPK3, MAPKAPK5, MAPKAPKps1, MARK1, MARK2, MARK3, MARK4, MARKps01, MARKps02, MARKps03, MARKps04, MARKps05, MARKps07, MARKps08, MARKps09, MARKps10, MARKps11, MARKps 12, MARKps13, MARKps15, MARKps16, MARKps17, MARKps18, MARKps19, MARKps20, MARKps21, MARKps22, MARKps23, MARKps24, MARKps25, MARKps26, MARKps27, MARKps28, MARKps29, MARKps30, MAST1, MAST2, MAST3, MAST4, MASTL, MELK, MER, MET, MISR2, MLK1, MLK2, MLK3, MLK4, MLKL, MNK1, MNK1ps, MNK2, MOK, MOS, MPSK1, MPSK1ps, MRCKa, MRCKb, MRCKps, MSK1, MSK12, MSK2, MSK22, MSSK1, MST1, MST2, MST3, MST3ps, MST4, MUSK, MYO3A, MYO3B, MYT1, NDR1, NDR2, NEK1, NEK10, NEK11, NEK2, NEK2ps1, NEK2ps2, NEK2ps3, NEK3, NEK4, NEK4ps, NEK5, NEK6, NEK7, NEK8, NEK9, NIK, NIM1, NLK, NRBP1, NRBP2, NuaK1, NuaK2, Obscn, Obscn2, OSR1, p38a, p38b, p38d, p38g, p70S6K, p70S6Kb, p70S6Kps1, p70S6Kps2, PAK1, PAK2, PAK2ps, PAK3, PAK4, PAK5, PAK6, PASK, PBK, PCTAIRE1, PCTAIRE2, PCTAIRE3, PDGFRa, PDGFRb, PDK1, PEK, PFTAIRE1, PFTAIRE2, PHKg1, PHKg1ps1, PHKg1ps2, PHKg1ps3, PHKg2, PIK3R4, PIKfyve, PIM1, PIM2, PIM3, PINK1, PITSLRE, PKACa, PKACb, PKACg, PKCa, PKCb, PKCd, PKCe, PKCg, PKCh, PKCi, PKCips, PKCt, PKCz, PKD1, PKD2, PKD3, PKG1, PKG2, PKN1, PKN2, PKN3, PKR, PLK1, PLK1ps1, PLK1ps2, PLK2, PLK3, PLK4, PRKX, PRKXps, PRKY, PRP4, PRP4ps, PRPK, PSKH1, PSKH1ps, PSKH2, PYK2, QIK, QSK, RAF1, RAF1ps, RET, RHOK, RIPK1, RIPK2, RIPK3, RNAseL, ROCK1, ROCK2, RON, ROR1, ROR2, ROS, RSK1, RSK12, RSK2, RSK22, RSK3, RSK32, RSK4, RSK42, RSKL1, RSKL2, RYK, RYKps, SAKps, SBK, SCYL1, SCYL2, SCYL2ps, SCYL3, SGK, SgK050ps, SgK069, SgK071, SgK085, SgK110, SgK196, SGK2, SgK223, SgK269, SgK288, SGK3, SgK307, SgK384ps, SgK396, SgK424, SgK493, SgK494, SgK495, SgK496, SIK (e.g., SIK1, SIK2), skMLCK, SLK, Slob, smMLCK, SNRK, SPEG, SPEG2, SRC, SRM, SRPK1, SRPK2, SRPK2ps, SSTK, STK33, STK33ps, STLK3, STLK5, STLK6, STLK6ps1, STLK6-rs, SuRTK106, SYK, TAK1, TAO1, TAO2, TAO3, TBCK, TBK1, TEC, TESK1, TESK2, TGFbR1, TGFbR2, TIE1, TIE2, TLK1, TLK1ps, TLK2, TLK2ps1, TLK2ps2, TNK1, Trad, Trb1, Trb2, Trb3, Trio, TRKA, TRKB, TRKC, TSSK1, TSSK2, TSSK3, TSSK4, TSSKps1, TSSKps2, TTBK1, TTBK2, TTK, TTN, TXK, TYK2, TYK22, TYRO3, TYRO3ps, ULK1, ULK2, ULK3, ULK4, VACAMKL, VRK1, VRK2, VRK3, VRK3ps, Wee1, Wee1B, Wee1Bps, Wee1ps1, Wee1ps2, Wnk1, Wnk2, Wnk3, Wnk4, YANK1, YANK2, YANK3, YES, YESps, YSK1, ZAK, ZAP70, ZC1/HGK, ZC2/TNIK, ZC3/MINK, and ZC4/NRK.

“PIKfyve” or “PIKfyve kinase” is a FYVE finger-containing phosphoinositide kinase. PIKfyve plays a role in cell entry of viruses after endocytosis.

A “serine protease” is a type of enzyme that cleaves peptide bonds in proteins, Serine proteases contain a catalytic triad consisting of three amino acids: His 57, Ser 195, and Asp 102. Serine proteases are involved in extracellular proteolysis.

“Transmembrane serine protease 2” or “TMPRSS2” is a type of serine protease that contains a type II transmembrane domain, a receptor class A domain, a scavenger receptor cysteine-rich domain and a protease domain. TMPRSS2 also facilitates entry of viruses into host cells by proteolytically cleaving and activating viral envelope glycoproteins.

The term “about X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

“Apilimod” refers to N-[(E)-(3-methylphenyl)methylidencamino]-6-morpholin-4-yl-2-(2-pyridin-2-ylethoxy)pyrimidin-4-amine. “Apilimod dimesylate” refers to the dimethanesulfonate salt of apilimod.

“Nafamostat” refers to (6-carbamimidoylnaphthalen-2-yl) 4-diaminomethylidencamino)benzoate. “Nafamostat mesylate” refers to the dimethanesulfonate salt of nafamostat.

“Camostat” refers to [4-[2-[2-(dimethylamino)-2-oxocthoxy]-2-oxoethyl]phenyl] 4-(diaminomethylidencamino)benzoate. “Camostat mesylate” refers to the dimethanesulfonate salt of camostat.

### DETAILED DESCRIPTION OF CERTAIN EMBODIMENTS

Described herein are compositions (e.g., pharmaceutical compositions) comprising a phosphatidylinositol-3-phosphate 5-kinase (PIKfyve kinase) inhibitor, or a pharmaceutically acceptable salt, solvate, hydrate, stereoisomer, polymorph, tautomer, isotopically enriched form, or prodrug thereof; a serine protease inhibitor, or a pharmaceutically acceptable salt, solvate, hydrate, stereoisomer, polymorph, tautomer, isotopically enriched form, or prodrug thereof; and water. The compositions may be useful in treating and/or preventing a viral infection (e.g., infection by a coronavirus, an ebolavirus, a Lassa virus) in a subject. The compositions may be useful in treating and/or preventing damage to respiratory tissue (e.g. upper respiratory tissue, for example, nasal tissue, nasopharyngeal tissue, and/or lower respiratory tissue, for example, lung tissue) in a subject. Also provided in the present disclosure are methods, uses, and kits involving the compositions described herein. The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compositions

The present disclosure provides compositions (e.g., pharmaceutical compositions) comprising a phosphatidylinositol-3-phosphate 5-kinase (PIKfyve kinase) inhibitor, or a pharmaceutically acceptable salt, solvate, hydrate, stereoisomer, polymorph, tautomer, isotopically enriched form, or prodrug thereof; a serine protease (e.g. TMPRSS2) inhibitor, or a pharmaceutically acceptable salt, solvate, hydrate, stereoisomer, polymorph, tautomer, isotopically enriched form, or prodrug thereof; and water. In certain embodiments, the composition is a pharmaceutical composition.

In certain embodiments, the subject is an animal. The animal may be of either sex. The animal may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate, In certain embodiments, the animal is a genetically engineered animal. In certain embodiments, the animal is a transgenic animal (e.g., transgenic mice and transgenic pigs). In certain embodiments, the subject is a fish or reptile,

In certain embodiments, the cell being contacted with a compound or pharmaceutical composition thereof described herein is in vitro. In certain embodiments, the cell being contacted with a compound or pharmaceutical composition thereof described herein is in vivo,

Pharmaceutical compositions described herein can be prepared by any method known in the art of pharmacology. In general, such preparatory methods include bringing the compound(s) described herein (i.e., the “active ingredient(s)”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0,1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly (vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof.

Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for intranasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for case of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are intranasal administration, oral administration, intravenous administration (c,g,, systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject. In certain embodiments, the compound or composition is administered intradermally, intramuscularly, intravaginally, intravenously, intranasally, orally, subcutaneously, topically, and/or sublingually.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample (e.g., tissue, cell), any two doses of the multiple doses include different or substantially the same amounts of a compound described herein. In certain embodiments, when multiple doses are administered to a subject or applied to a biological sample (e.g., tissue, cell), the frequency of administering the multiple doses to the subject or applying the multiple doses to the biological sample (e.g., tissue, cell) is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample (e.g., tissue, cell) is one dose per day. In certain embodiments, the frequency of administering the multiple doses to the subject or applying the multiple doses to the biological sample (e.g., tissue, cell) is two doses per day. In certain embodiments, the frequency of administering the multiple doses to the subject or applying the multiple doses to the biological sample (e.g., tissue, cell) is three doses per day. In certain embodiments, the frequency of administering the multiple doses to the subject or applying the multiple doses to the biological sample (e.g., tissue, cell) is four doses per day. In certain embodiments, the frequency of administering the multiple doses to the subject or applying the multiple doses to the biological sample (e.g., tissue, cell) is five doses per day. In certain embodiments, the frequency of administering the multiple doses to the subject or applying the multiple doses to the biological sample (e.g., tissue, cell) is one dose every hour. In certain embodiments, the frequency of administering the multiple doses to the subject or applying the multiple doses to the biological sample (e.g., tissue, cell) is one dose every two hours. In certain embodiments, the frequency of administering the multiple doses to the subject or applying the multiple doses to the biological sample (e.g., tissue, cell) is one dose every three hours. In certain embodiments, the frequency of administering the multiple doses to the subject or applying the multiple doses to the biological sample (e.g., tissue, cell) is one dose every four hours. In certain embodiments, the frequency of administering the multiple doses to the subject or applying the multiple doses to the biological sample (e.g., tissue, cell) is one dose every six hours. In certain embodiments, the frequency of administering the multiple doses to the subject or applying the multiple doses to the biological sample (e.g., tissue, cell) is one dose every eight hours. In certain embodiments, the frequency of administering the multiple doses to the subject or applying the multiple doses to the biological sample (e.g., tissue, cell) is one dose every twelve hours. In certain embodiments, when multiple doses are administered to a subject or applied to a biological sample (e.g., tissue, cell), the duration between the first dose and last dose of the multiple doses is one day, two days, three days, four days, five days, six days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein, In certain embodiments, a dose described herein includes independently between 10 mg and 30 mg, inclusive, of a compound described herein. In certain embodiments, a dose described herein includes independently between 30 mg and 100 mg, inclusive, of a compound described herein. In certain embodiments, a dose described herein includes independently between 100 mg and 300 mg, inclusive, of a compound described herein. In certain embodiments, a dose described herein includes independently between 300 mg and 1,000 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or pharmaceutical composition thereof, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in inhibiting, for example, inhibiting the activity of PIKfyve kinase or a serine protease (e.g., TMPRSS2), improve bioavailability, improve safety, reduce drug resistance, reduce and/or modify metabolism, inhibit excretion, and/or modify distribution in a subject and/or biological sample (e.g., tissue, cell). It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compounds described herein and the additional pharmaceutical agent, but not both.

The compound or pharmaceutical composition thereof can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, inflammatory disease, autoimmune disease). Each additional pharmaceutical agent may be administered at a dose and/or on a time schedule determined for that pharmaceutical agent. The additional pharmaceutical agents may also be administered together with each other and/or with the compound or pharmaceutical composition thereof described herein in a single dose or administered separately in different doses. The particular combination to employ in a regimen will take into account compatibility of the compound described herein with the additional pharmaceutical agent(s) and/or the desired therapeutic and/or prophylactic effect to be achieved.

In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof.

In certain embodiments, the serine protease inhibitor inhibits transmembrane serine protease 2 (TMPRSS2).

In certain embodiments, the PIKfyve kinase inhibitor is apilimod (e.g. apilimod dimesylate), vacuolin-1 (3-iodo-benzaldehyde,2-[4-(diphenylamino)-6-(4-morpholinyl)-1,3,5-triazin-2-yl] hydrazone), YM-201636 (6-amino-N-[3-[4-(4-morpholinyl)pyrido[3′,2′:4,5]furo[3,2-d]pyrimidin-2-yl]phenyl]-3-pyridinecarboxamide), APY0201 (2-[7-(4-morpholinyl)-2-(4-pyridinyl)pyrazolo[1,5-a]pyrimidin-5-yl]hydrazone, 3-methyl-benzaldehyde), MOMIPP (3-(5-methoxy, 2-methyl-1H-indol-3-yl)-1-(4-pyridinyl)-2-propen-1-one), WX8 (1H-indole-3-carbaldehyde [4-anilino-6-(4-morpholinyl)-1,3,5-triazin-2-yl]hydrazine), NDF (3-methylbenzaldehyde (2,6-dimorpholin-4-ylpyrimidin-4-yl)hydrazine), WWL (benzaldehyde [2,6-di(4-morpholinyl)-4-pyrimidinyl]hydrazone), XB6 (N-(4-ethylphenyl)-4,6-dimorpholino-1,3,5-triazin-2-amine hydrochloride), XBA (9 N-(3-chloro-4-fluorophenyl)-4,6-dimorpholino-1,3,5-triazin-2-amine hydrochloride), or a pharmaceutically acceptable salt thereof. In certain embodiments, the PIKfyve kinase inhibitor is apilimod (e.g. apilimod dimesylate) or vacuolin-1, or a pharmaceutically acceptable salt thereof. In certain embodiments, the PIKfyve kinase inhibitor is apilimod (e.g. apilimod dimesylate), or a pharmaceutically acceptable salt thereof. In certain embodiments, the PIKfyve kinase inhibitor is apilimod dimesylate. In certain embodiments, the PIKfyve kinase inhibitor is vacuolin-1, or a pharmaceutically acceptable salt thereof.

In certain embodiments, the composition comprises about 1-100 mg/mL apilimod dimesylate. In certain embodiments, the composition comprises about 1-50 mg/mL or about 50-100 mg/mL apilimod dimesylate. In certain embodiments, the composition comprises about 1-50 mg/mL apilimod dimesylate. In certain embodiments, the composition comprises about 50-100 mg/mL apilimod dimesylate. In certain embodiments, the composition comprises about 1-25 mg/mL, about 25-50 mg/mL, about 50-75 mg/mL, or about 75-100 mg/mL apilimod dimesylate. In certain embodiments, the composition comprises about 1-25 mg/mL apilimod dimesylate. In certain embodiments, the composition comprises about 25-50 mg/mL apilimod dimesylate. In certain embodiments, the composition comprises about 50-75 mg/mL apilimod dimesylate. In certain embodiments, the composition comprises about 75-100 mg/mL apilimod dimesylate. In certain embodiments, the composition comprises about 1-20 mg/mL, about 20-40 mg/mL, about 40-60 mg/mL, about 60-80 mg/mL, or about 80-100 mg/mL apilimod dimesylate. In certain embodiments, the composition comprises about 1-20 mg/mL apilimod dimesylate. In certain embodiments, the composition comprises about 20-40 mg/mL apilimod dimesylate. In certain embodiments, the composition comprises about 40-60 mg/mL apilimod dimesylate. In certain embodiments, the composition comprises about 60-80 mg/mL apilimod dimesylate. In certain embodiments, the composition comprises about 80-100 mg/mL apilimod dimesylate. In certain embodiments, the composition comprises about 1-10 mg/mL, about 10-20 mg/mL, about 20-30 mg/mL, about 30-40 mg/mL, about 40-50 mg/mL, about 50-60 mg/mL, about 60-70 mg/mL, about 70-80 mg/mL, about 80-90 mg/mL, or about 90-100 mg/mL apilimod dimesylate. In certain embodiments, the composition comprises about 1-10 mg/mL apilimod dimesylate. In certain embodiments, the composition comprises about 10-20 mg/mL (e.g. about 18 mg/mL) apilimod dimesylate. In certain embodiments, the composition comprises about 20-30 mg/mL apilimod dimesylate. In certain embodiments, the composition comprises about 30-40 mg/mL apilimod dimesylate. In certain embodiments, the composition comprises about 40-50 mg/mL apilimod dimesylate. In certain embodiments, the composition comprises about 50-60 mg/mL apilimod dimesylate. In certain embodiments, the composition comprises about 60-70 mg/mL apilimod dimesylate. In certain embodiments, the composition comprises about 70-80 mg/mL apilimod dimesylate. In certain embodiments, the composition comprises about 80-90 mg/mL apilimod dimesylate. In certain embodiments, the composition comprises about 90-100 mg/mL apilimod dimesylate. In certain embodiments, the composition comprises about 10-30 mg/mL apilimod dimesylate. In certain embodiments, the composition comprises about 15-20 mg/mL (e.g., 18 mg/mL) apilimod dimesylate. In certain embodiments, the composition comprises about 18 mg/mL apilimod dimesylate.

In certain embodiments, the serine protease inhibitor is nafamostat (e.g., nafamostat mesylate) or camostat (e.g. camostat mesylate), or a pharmaceutically acceptable salt thereof. In certain embodiments, the serine protease inhibitor is nafamostat (e.g., nafamostat mesylate). In certain embodiments, the serine protease inhibitor is nafamostat mesylate. In certain embodiments, the serine protease inhibitor is camostat (e.g. camostat mesylate). In certain embodiments, the serine protease inhibitor is nafamostat mesylate or camostat mesylate.

In certain embodiments, the composition comprises about 1-200 mg/mL nafamostat mesylate. In certain embodiments, the composition comprises about 1-100 mg/mL or about 100-200 mg/mL nafamostat mesylate. In certain embodiments, the composition comprises about 1-100 mg/mL nafamostat mesylate. In certain embodiments, the composition comprises about 100-200 mg/mL nafamostat mesylate.

In certain embodiments, the composition comprises about 1-50 mg/mL, about 50-100 mg/mL, about 100-150 mg/mL, or about 150-200 mg/mL nafamostat mesylate. In certain embodiments, the composition comprises about 1-50 mg/mL nafamostat mesylate. In certain embodiments, the composition comprises about 50-100 mg/mL nafamostat mesylate. In certain embodiments, the composition comprises about 100-150 mg/mL nafamostat mesylate. In certain embodiments, the composition comprises about 150-200 mg/mL nafamostat mesylate.

In certain embodiments, the composition comprises about 1-25 mg/mL, about 25-50 mg/mL, about 50-75 mg/mL, about 75-100 mg/mL, about 100-125 mg/mL, about 125-150 mg/mL, about 150-175 mg/mL, or about 175-200 mg/mL nafamostat mesylate. In certain embodiments, the composition comprises about 1-25 mg/mL nafamostat mesylate. In certain embodiments, the composition comprises about 25-50 mg/mL nafamostat mesylate. In certain embodiments, the composition comprises about 50-75 mg/mL nafamostat mesylate. In certain embodiments, the composition comprises about 75-100 mg/mL nafamostat mesylate. In certain embodiments, the composition comprises about 100-125 mg/mL nafamostat mesylate. In certain embodiments, the composition comprises about 125-150 mg/mL nafamostat mesylate. In certain embodiments, the composition comprises about 150-175 mg/mL nafamostat mesylate. In certain embodiments, the composition comprises about 175-200 mg/mL nafamostat mesylate.

In certain embodiments, the composition comprises about 1-20 mg/mL, about 20-40 mg/mL, about 40-60 mg/mL, about 60-80 mg/mL, about 80-100 mg/mL, about 100-120 mg/mL, about 120-140 mg/mL, about 140-160 mg/mL, about 160-180 mg/mL, or about 180-200 mg/mL nafamostat mesylate. In certain embodiments, the composition comprises about 1-20 mg/mL nafamostat mesylate. In certain embodiments, the composition comprises about 20-40 mg/mL (e.g. about 36 mg/mL) nafamostat mesylate. In certain embodiments, the composition comprises about 40-60 mg/mL nafamostat mesylate. In certain embodiments, the composition comprises about 60-80 mg/mL nafamostat mesylate. In certain embodiments, the composition comprises about 80-100 mg/mL nafamostat mesylate. In certain embodiments, the composition comprises about 100-20 mg/mL nafamostat mesylate. In certain embodiments, the composition comprises about 120-140 mg/mL nafamostat mesylate. In certain embodiments, the composition comprises about 140-160 mg/mL nafamostat mesylate. In certain embodiments, the composition comprises about 160-180 mg/mL nafamostat mesylate. In certain embodiments, the composition comprises about 180-200 mg/mL nafamostat mesylate.

In certain embodiments, the composition comprises about 1-10 mg/mL, about 10-20 mg/mL, about 20-30 mg/mL, about 30-40 mg/mL, about 40-50 mg/mL, about 50-60 mg/mL, about 60-70 mg/mL, about 70-80 mg/mL, about 80-90 mg/mL, about 90-100 mg/mL, about 100-110 mg/mL, about 110-120 mg/mL, about 120-130 mg/mL, about 130-140 mg/mL, about 140-150 mg/mL, about 150-160 mg/mL, about 160-170 mg/mL, about 170-180 mg/mL, about 180-190 mg/mL, or about 190-200 mg/mL nafamostat mesylate. In certain embodiments, the composition comprises about 1-10 mg/mL nafamostat mesylate. In certain embodiments, the composition comprises about 10-20 mg/mL nafamostat mesylate. In certain embodiments, the composition comprises about 20-30 mg/mL nafamostat mesylate. In certain embodiments, the composition comprises about 30-40 mg/mL (e.g. about 36 mg/mL) nafamostat mesylate. In certain embodiments, the composition comprises about 40-50 mg/mL nafamostat mesylate. In certain embodiments, the composition comprises about 50-60 mg/mL nafamostat mesylate. In certain embodiments, the composition comprises about 60-70 mg/mL nafamostat mesylate. In certain embodiments, the composition comprises about 70-80 mg/mL nafamostat mesylate. In certain embodiments, the composition comprises about 80-90 mg/mL nafamostat mesylate. In certain embodiments, the composition comprises about 90-100 mg/mL nafamostat mesylate. In certain embodiments, the composition comprises about 100-110 mg/mL nafamostat mesylate. In certain embodiments, the composition comprises about 110-120 mg/mL nafamostat mesylate. In certain embodiments, the composition comprises about 120-130 mg/mL nafamostat mesylate. In certain embodiments, the composition comprises about 130-140 mg/mL nafamostat mesylate. In certain embodiments, the composition comprises about 140-150 mg/mL nafamostat mesylate. In certain embodiments, the composition comprises about 150-160 mg/mL nafamostat mesylate. In certain embodiments, the composition comprises about 160-710 mg/mL nafamostat mesylate. In certain embodiments, the composition comprises about 170-180 mg/mL nafamostat mesylate. In certain embodiments, the composition comprises about 180-190 mg/mL nafamostat mesylate. In certain embodiments, the composition comprises about 190-200 mg/mL nafamostat mesylate. In certain embodiments, the composition comprises about 10-50 mg/mL nafamostat mesylate. In certain embodiments, the composition comprises about 30-40 mg/mL (e.g., about 36 mg/mL) nafamostat mesylate. In certain embodiments, the composition comprises about 36 mg/mL nafamostat mesylate.

In certain embodiments, the composition comprises apilimod (e.g. apilimod dimesylate) and nafamostat (e.g., nafamostat mesylate). In certain embodiments, the composition comprises apilimod dimesylate and nafamostat (e.g., nafamostat mesylate). In certain embodiments, the composition comprises apilimod (e.g. apilimod dimesylate) and nafamostat mesylate. In certain embodiments, the composition comprises apilimod dimesylate and nafamostat mesylate.

In certain embodiments, the composition comprises about 1-100 mg/mL apilimod dimesylate (e.g. about 1-50 mg/mL or about 50-100 mg/mL) and about 1-200 mg/mL nafamostat mesylate (e.g. about 1-100 mg/mL or 100-200 mg/mL). In certain embodiments, the composition comprises about 1-50 mg/mL apilimod dimesylate and about 1-100 mg/ml nafamostat mesylate. In certain embodiments, the composition comprises about 50-100 mg/mL apilimod dimesylate and about 100-200 mg/mL nafamostat mesylate.

In certain embodiments, the composition comprises about 1-100 mg/mL apilimod dimesylate (e.g. about 1-25 mg/mL, about 25-50 mg/mL, about 50-75 mg/mL or about 75-100 mg/mL) and about 1-200 mg/mL nafamostat mesylate (e.g. about 1-50 mg/mL, about 50-100 mg/mL, about 100-150 mg/mL, or about 150-200 mg/mL). In certain embodiments, the composition comprises about 1-25 mg/mL apilimod dimesylate and about 1-50 mg/mL nafamostat mesylate. In certain embodiments, the composition comprises about 25-50 mg/mL apilimod dimesylate and about 50-100 mg/mL nafamostat mesylate. In certain embodiments, the composition comprises about 50-75 mg/mL apilimod dimesylate and about 100-150 mg/mL nafamostat mesylate. In certain embodiments, the composition comprises about 75-100 mg/mL apilimod dimesylate and about 150-200 mg/mL nafamostat mesylate.

In certain embodiments, the composition comprises about 1-100 mg/mL apilimod dimesylate (e.g. about 1-20 mg/mL, about 20-40 mg/mL, about 40-60 mg/mL, about 60-80 mg/mL, or about 80-100 mg/mL) and about 1-200 mg/mL nafamostat mesylate (e.g. about 1-40 mg/mL, about 40-80 mg/mL, about 80-120 mg/mL, about 120-160 mg/mL, or about 160-200 mg/mL). In certain embodiments, the composition comprises about 1-20 mg/mL apilimod dimesylate and about 1-40 mg/mL nafamostat mesylate. In certain embodiments, the composition comprises about 20-40 mg/mL apilimod dimesylate and about 40-80 mg/mL nafamostat mesylate. In certain embodiments, the composition comprises about 40-60 mg/ml apilimod dimesylate and about 80-120 mg/mL nafamostat mesylate. In certain embodiments, the composition comprises about 60-80 mg/mL apilimod dimesylate and about 120-160 mg/mL nafamostat mesylate. In certain embodiments, the composition comprises about 80-100 mg/mL apilimod dimesylate and about 160-200 mg/mL nafamostat mesylate.

In certain embodiments, the composition comprises about 1-100 mg/mL apilimod dimesylate (e.g. about 1-10 mg/mL, about 10-20 mg/mL, about 20-30 mg/mL, about 30-40 mg/mL, about 40-50 mg/mL, about 50-60 mg/mL, about 60-70 mg/mL, about 70-80 mg/mL, about 80-90 mg/mL, or about 90-100 mg/mL) and about 1-200 mg/mL nafamostat mesylate (e.g. about 1-20 mg/mL, about 20-40 mg/mL, about 40-60 mg/mL, about 60-80 mg/mL, about 80-100 mg/mL, about 100-120 mg/mL, about 120-140 mg/mL, about 140-160 mg/mL, about 160-180 mg/mL, or about 180-200 mg/mL). In certain embodiments, the composition comprises about 1-10 mg/mL apilimod dimesylate and about 1-20 mg/mL nafamostat mesylate. In certain embodiments, the composition comprises about 10-20 mg/mL (e.g. about 18 mg/mL) apilimod dimesylate and about 20-40 mg/mL (e.g. about 36 mg/mL) nafamostat mesylate. In certain embodiments, the composition comprises about 20-30 mg/mL apilimod dimesylate and about 40-60 mg/mL nafamostat mesylate. In certain embodiments, the composition comprises about 30-40 mg/mL apilimod dimesylate and about 60-80 mg/mL nafamostat mesylate. In certain embodiments, the composition comprises about 40-50 mg/mL apilimod dimesylate and about 80-100 mg/mL nafamostat mesylate. In certain embodiments, the composition comprises about 50-60 mg/mL apilimod dimesylate and about 100-120 mg/mL nafamostat mesylate. In certain embodiments, the composition comprises about 60-70 mg/mL apilimod dimesylate and about 120-140 mg/mL nafamostat mesylate. In certain embodiments, the composition comprises about 70-80 mg/mL apilimod dimesylate and about 140-160 mg/mL nafamostat mesylate. In certain embodiments, the composition comprises about 80-90 mg/mL apilimod dimesylate and about 160-180 mg/mL nafamostat mesylate. In certain embodiments, the composition comprises about 90-100 mg/mL apilimod dimesylate and about 180-200 mg/mL nafamostat mesylate.

In certain embodiments, the composition comprises about 10-30 mg/mL apilimod dimesylate and about 20-60 mg/mL nafamostat mesylate. In certain embodiments, the composition comprises about 15-20 mg/mL (e.g. about 18 mg/mL) apilimod dimesylate and about 30-40 mg/mL (e.g. about 36 mg/mL) nafamostat mesylate. In certain embodiments, the composition comprises about 18 mg/mL apilimod dimesylate and about 36 mg/mL nafamostat mesylate.

In certain embodiments, the composition comprises about 1-400 mg apilimod dimesylate. In certain embodiments, the composition comprises about 1-200 mg or about 200-400 mg apilimod dimesylate. In certain embodiments, the composition comprises about 1-100 mg, about 100-200 mg, about 200-300 mg, or about 300-400 mg apilimod dimesylate. In certain embodiments, the composition comprises about 1-50 mg, about 50-100 mg, about 100-150 mg, about 150-200 mg, about 200-250 mg, about 250-300 mg, about 300-350 mg, or about 350-400 mg apilimod dimesylate. In certain embodiments, the composition comprises about 1-25 mg, about 25-50 mg, about 50-75 mg, about 75-100 mg, about 100-125 mg, about 125-150 mg, about 150-175 mg, about 175-200 mg, about 200-225 mg, about 225-250 mg, about 250-275 mg, about 275-300 mg, about 300-325 mg, about 325-350 mg, about 350-375 mg, or about 375-400 mg apilimod dimesylate.

In certain embodiments, the composition comprises about 1-400 mg nafamostat mesylate. In certain embodiments, the composition comprises about 1-200 mg or about 200-400 mg nafamostat mesylate. In certain embodiments, the composition comprises about 1-100 mg, about 100-200 mg, about 200-300 mg, or about 300-400 mg nafamostat mesylate. In certain embodiments, the composition comprises about 1-50 mg, about 50-100 mg, about 100-150 mg, about 150-200 mg, about 200-250 mg, about 250-300 mg, about 300-350 mg, or about 350-400mg nafamostat mesylate. In certain embodiments, the composition comprises about 1-25 mg, about 25-50 mg, about 50-75 mg, about 75-100 mg, about 100-125 mg, about 125-150 mg, about 150-175 mg, about 175-200 mg, about 200-225 mg, about 225-250 mg, about 250-275 mg, about 275-300 mg, about 300-325 mg, about 325-350 mg, about 350-375 mg, or about 375-400 mg nafamostat mesylate.

In certain embodiments, the composition comprises about 1-400 mg apilimod dimesylate (e.g. about 1-100 mg, about 100-200 mg, about 200-300 mg, or about 300-400 mg) and about 1-400 mg nafamostat mesylate (e.g. about 1-100 mg, about 100-200 mg, about 200-300 mg, or about 300-400 mg). In certain embodiments, the composition comprises about 1-400 mg apilimod dimesylate (e.g. about 1-50 mg, about 50-100 mg, about 100-150 mg, about 150-200 mg, about 200-250 mg, about 250-300 mg, about 300-350 mg, or about 350-400 mg) and about 1-400 mg nafamostat mesylate (e.g. about 1-50 mg, about 50-100 mg, about 100-150 mg, about 150-200 mg, about 200-250 mg, about 250-300 mg, about 300-350 mg, or about 350-400 mg).

In certain embodiments, the composition comprises about 1-400 mg apilimod dimesylate (e.g. about 1-25 mg, about 25-50 mg, about 50-75 mg, about 75-100 mg, about 100-125 mg, about 125-150 mg, about 150-175 mg, about 175-200 mg, about 200-225 mg, about 225-250 mg, about 250-275 mg, about 275-300 mg, about 300-325 mg, about 325-350 mg, about 350-375 mg, or about 375-400 mg) and about 1-400 mg nafamostat mesylate (e.g. about 1-25 mg, about 25-50 mg, about 50-75 mg, about 75-100 mg, about 100-125 mg, about 125-150 mg, about 150-175 mg, about 175-200 mg, about 200-225 mg, about 225-250 mg, about 250-275 mg, about 275-300 mg, about 300-325 mg, about 325-350 mg, about 350-375 mg, or about 375-400 mg). In certain embodiments, the composition further comprises an additional solvent. In certain embodiments, the additional solvent is a polar aprotic solvent. In certain embodiments, the polar aprotic solvent is dimethylsulfoxide (DMSO), an acetate-containing solvent (e.g. methyl acetate, ethyl acetate), acetonitrile, or dimethylformamide. In certain embodiments, the polar aprotic solvent is dimethylsulfoxide (DMSO). In certain embodiments, the additional solvent is an alcohol. In certain embodiments, the alcohol is benzyl alcohol, ethanol, isopropanol, polyethylene glycol, phenol, phenolic compounds, bisphenol, chlorobutanol, hydroxybenzoate, or phenylethyl alcohol. In certain embodiments, the alcohol is ethanol.

In certain embodiments, the composition comprises a solution of a PIKfyve kinase inhibitor, or a pharmaceutically acceptable salt, solvate, hydrate, stereoisomer, polymorph, tautomer, isotopically enriched form, or prodrug thereof; a serine protease inhibitor, or a pharmaceutically acceptable salt, solvate, hydrate, stereoisomer, polymorph, tautomer, isotopically enriched form, or prodrug thereof; and water.

In certain embodiments, the water is deionized water. In certain embodiments, the deionized water has a salt concentration of less than about 200 mM (e.g. less than about 175 mM, less than about 150 mM, less than about 125 mM, less than about 100 mM, less than about 75 mM, less than about 50 mM, less than about 25 mM). In certain embodiments, the deionized water has a salt concentration of less than about 175 mM (e.g. less than about 150 mM, less than about 125 mM, less than about 100 mM, less than about 75 mM, less than about 50 mM, less than about 25 mM). In certain embodiments, the deionized water has a salt concentration of less than about 150 mM (e.g. less than about 125 mM, less than about 100 mM, less than about 75 mM, less than about 50 mM, less than about 25 mM). In certain embodiments, the deionized water has a salt concentration of less than about 125 mM (e.g. less than about 100 mM, less than about 75 mM, less than about 50 mM, less than about 25 mM). In certain embodiments, the deionized water has a salt concentration of less than about 100 mM (e.g. less than about 75 mM, less than about 50 mM, less than about 25 mM). In certain embodiments, the deionized water has a salt concentration of less than about 75 mM (e.g. less than about 50 mM, less than about 25 mM). In certain embodiments, the deionized water has a salt concentration of less than about 50 mM (e.g. less than about 25 mM). In certain embodiments, the deionized water has a salt concentration of less than about 25 mM. In certain embodiments, the water is distilled water. In certain embodiments, the water is tap water.

In certain embodiments, the composition has a pH of about 2.0-12.0. In certain embodiments, the composition has a pH of about 2.0-8.0. In certain embodiments, the composition has a pH of about 2.0-7.0. In certain embodiments, the composition has a pH of about 2.0-5.0. In certain embodiments, the composition has a pH of about 3.0-4.5. In certain embodiments, the composition has a pH of about 3.3. In certain embodiments, the composition has a pH of about 3.6. In certain embodiments, the composition has a pH of about 4.1. In certain embodiments, the composition has a pH of about 7.0-12.0. In certain embodiments, the composition has a pH of about 7.0-10.0. In certain embodiments, the intranasal pH is about 6.2-6.8.

In certain embodiments, the composition further comprises a sugar (e.g., sucrose). In certain embodiments, the composition further comprises sucrose.

In certain embodiments, the composition is stable for at least one day, at least two days, at least three days, at least four days, at least five days, at least six days, at least one week, at least two weeks, at least three weeks, at least 1 month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years. In certain embodiments, the composition is stable for at least one day. In certain embodiments, the composition is stable for at least two days. In certain embodiments, the composition is stable for at least three days. In certain embodiments, the composition is stable for at least four days. In certain embodiments, the composition is stable for at least five days. In certain embodiments, the composition is stable for at least six days. In certain embodiments, the composition is stable for at least one week. In certain embodiments, the composition is stable for at least two weeks. In certain embodiments, the composition is stable for at least three weeks. In certain embodiments, the composition is stable for at least 1 month. In certain embodiments, the composition is stable for at least 2 months. In certain embodiments, the composition is stable for at least 3 months. In certain embodiments, the composition is stable for at least 4 months. In certain embodiments, the composition is stable for at least 5 months. In certain embodiments, the composition is stable for at least 6 months. In certain embodiments, the composition is stable for at least 7 months. In certain embodiments, the composition is stable for at least 8 months. In certain embodiments, the composition is stable for at least 9 months. In certain embodiments, the composition is stable for at least 10 months. In certain embodiments, the composition is stable for at least 11 months. In certain embodiments, the composition is stable for at least 1 year. In certain embodiments, the composition is stable for at least 2 years. In certain embodiments, the composition is stable for at least 3 years.

In certain embodiments, the composition is stable for at least one day at room temperature (e.g., about 20-40° C.), at least two days at room temperature (e.g., about 20-40° C.), at least three days at room temperature (e.g., about 20-40° C.), at least four days at room temperature (e.g., about 20-40° C.), at least five days at room temperature (e.g., about 20-40° C.), at least six days at room temperature (e.g., about 20-40° C.), at least one week at room temperature (e.g., about 20-40° C.), at least two weeks at room temperature (e.g., about 20-40° C.), at least three weeks at room temperature (e.g., about 20-40° C.), at least 1 month at room temperature (e.g., about 20-40° C.), at least 2 months at room temperature (e.g., about 20-40° C.), at least 3 months at room temperature (e.g., about 20-40° C.), at least 4 months at room temperature (e.g., about 20-40° C.), at least 5 months at room temperature (e.g., about 20-40° C.), at least 6 months at room temperature (e.g., about 20-40° C.), at least 7 months at room temperature (e.g., about 20-40° C.), at least 8 months at room temperature (e.g., about 20-40° C.), at least 9 months at room temperature (e.g., about 20-40° C.), at least 10 months at room temperature (e.g., about 20-40° C.), at least 11 months at room temperature (e.g., about 20-40° C.), at least 1 year at room temperature (e.g., about 20-40° C.), at least 2 years at room temperature (e.g., about 20-40° C.), or at least 3 years at room temperature (e.g., about 20-40° C.). In certain embodiments, the composition is stable for at least one day at room temperature (e.g., about 20-40° C.). In certain embodiments, the composition is stable for at least two days at room temperature (e.g., about 20-40° C.). In certain embodiments, the composition is stable for at least three days at room temperature (e.g., about 20-40° C.). In certain embodiments, the composition is stable for at least four days at room temperature (e.g., about 20-40° C.). In certain embodiments, the composition is stable for at least five days at room temperature (e.g., about 20-40° C.). In certain embodiments, the composition is stable for at least six days at room temperature (e.g., about 20-40° C.). In certain embodiments, the composition is stable for at least one week at room temperature (e.g., about 20-40° C.). In certain embodiments, the composition is stable for at least two weeks at room temperature (e.g., about 20-40° C.). In certain embodiments, the composition is stable for at least three weeks at room temperature (e.g., about 20-40° C.). In certain embodiments, the composition is stable for at least 1 month at room temperature (e.g., about 20-40° C.). In certain embodiments, the composition is stable for at least 2 months at room temperature (e.g., about 20-40° C.). In certain embodiments, the composition is stable for at least 3 months at room temperature (e.g., about 20-40° C.). In certain embodiments, the composition is stable for at least 4 months at room temperature (e.g., about 20-40° C.). In certain embodiments, the composition is stable for at least 5 months at room temperature (e.g., about 20-40° C.). In certain embodiments, the composition is stable for at least 6 months at room temperature (e.g., about 20-40° C.). In certain embodiments, the composition is stable for at least 7 months at room temperature (e.g., about 20-40° C.). In certain embodiments, the composition is stable for at least 8 months at room temperature (e.g., about 20-40° C.). In certain embodiments, the composition is stable for at least 9 months at room temperature (e.g., about 20-40° C.). In certain embodiments, the composition is stable for at least 10 months at room temperature (e.g., about 20-40° C.). In certain embodiments, the composition is stable for at least 11 months at room temperature (e.g., about 20-40° C.). In certain embodiments, the composition is stable for at least 1 year at room temperature (e.g., about 20-40° C.). In certain embodiments, the composition is stable for at least 2 years at room temperature (e.g., about 20-40° C.). In certain embodiments, the composition is stable for at least 3 years at room temperature (e.g., about 20-40° C.).

In certain embodiments, the composition is stable at about 0-40° C. In certain embodiments, the composition is stable at about 0-20° C. In certain embodiments, the composition is stable at about 20-40° C. In certain embodiments, the composition is stable at about 0-10° C. In certain embodiments, the composition is stable at about 10-20° C. In certain embodiments, the composition is stable at about 20-30° C. In certain embodiments, the composition is stable at about 30-40° C. In certain embodiments, the composition is stable at about 0-5° C. In certain embodiments, the composition is stable at about 5-10° C. In certain embodiments, the composition is stable at about 10-15° C. In certain embodiments, the composition is stable at about 15-20° C. In certain embodiments, the composition is stable at about 20-25° C. In certain embodiments, the composition is stable at about 25-30° C. In certain embodiments, the composition is stable at about 30-35° C. In certain embodiments, the composition is stable at about 35-40° C. In certain embodiments, the composition is stable at about 4° C. In certain embodiments, the composition is stable at about 23,5° C. In certain embodiments, the composition is stable at about 37° C.

In certain embodiments, the composition retains antiviral efficacy for at least one day, at least two days, at least three days, at least four days, at least five days, at least six days, at least one week, at least two weeks, at least three weeks, at least 1 month, at least 2 months, at least 3 months, at least 4 months, at least 5 months, at least 6 months, at least 7 months, at least 8months, at least 9 months, at least 10 months, at least 11 months, at least 1 year, at least 2 years, or at least 3 years. In certain embodiments, the composition retains antiviral efficacy for at least one day. In certain embodiments, the composition retains antiviral efficacy for at least two days. In certain embodiments, the composition retains antiviral efficacy for at least three days. In certain embodiments, the composition retains antiviral efficacy for at least four days. In certain embodiments, the composition retains antiviral efficacy for at least five days. In certain embodiments, the composition retains antiviral efficacy for at least six days. In certain embodiments, the composition retains antiviral efficacy for at least one week. In certain embodiments, the composition retains antiviral efficacy for at least two weeks. In certain embodiments, the composition retains antiviral efficacy for at least three weeks. In certain embodiments, the composition retains antiviral efficacy for at least 1 month. In certain embodiments, the composition retains antiviral efficacy for at least 2 months. In certain embodiments, the composition retains antiviral efficacy for at least 3 months. In certain embodiments, the composition retains antiviral efficacy for at least 4 months. In certain embodiments, the composition retains antiviral efficacy for at least 5 months. In certain embodiments, the composition retains antiviral efficacy for at least 6 months. In certain embodiments, the composition retains antiviral efficacy for at least 7 months. In certain embodiments, the composition retains antiviral efficacy for at least 8 months. In certain embodiments, the composition retains antiviral efficacy for at least 9 months. In certain embodiments, the composition retains antiviral efficacy for at least 10 months. In certain embodiments, the composition retains antiviral efficacy for at least 11 months. In certain embodiments, the composition retains antiviral efficacy for at least 1 year. In certain embodiments, the composition retains antiviral efficacy for at least 2 years. In certain embodiments, the composition retains antiviral efficacy for at least 3 years. In certain embodiments, the composition retains antiviral efficacy for at least 5 years. In certain embodiments, the composition retains antiviral efficacy for at least 10 years.

In certain embodiments, the composition retains antiviral efficacy for at least one day at room temperature (e.g., about 20-40° C.), at least two days at room temperature (e.g., about 20-40° C.), at least three days at room temperature (e.g., about 20-40° C.), at least four days at room temperature (e.g., about 20-40° C.), at least five days at room temperature (e.g., about 20-40° C.), at least six days at room temperature (e.g., about 20-40° C.), at least one week at room temperature (e.g., about 20-40° C.), at least two weeks at room temperature (e.g., about 20-40° C.), at least three weeks at room temperature (e.g., about 20-40° C.), at least 1 month at room temperature (e.g., about 20-40° C.), at least 2 months at room temperature (e.g., about 20-40° C.), at least 3 months at room temperature (e.g., about 20-40° C.), at least 4 months at room temperature (e.g., about 20-40° C.), at least 5 months at room temperature (e.g., about 20-40° C.), at least 6 months at room temperature (e.g., about 20-40° C.), at least 7 months at room temperature (e.g., about 20-40° C.), at least 8 months at room temperature (e.g., about 20-40° C.), at least 9 months at room temperature (e.g., about 20-40° C.), at least 10 months at room temperature (e.g., about 20-40° C.), at least 11 months at room temperature (e.g., about 20-40° C.), at least 1 year at room temperature (e.g., about 20-40° C.), at least 2 years at room temperature (e.g., about 20-40° C.), or at least 3 years at room temperature (e.g., about 20-40° C.). In certain embodiments, the composition retains antiviral efficacy for at least one day at room temperature (e.g., about 20-40° C.). In certain embodiments, the composition retains antiviral efficacy for at least two days at room temperature (e.g., about 20-40° C.). In certain embodiments, the composition retains antiviral efficacy for at least three days at room temperature (e.g., about 20-40° C.). In certain embodiments, the composition retains antiviral efficacy for at least four days at room temperature (e.g., about 20-40° C.). In certain embodiments, the composition retains antiviral efficacy for at least five days at room temperature (e.g., about 20-40° C.). In certain embodiments, the composition retains antiviral efficacy for at least six days at room temperature (e.g., about 20-40° C.). In certain embodiments, the composition retains antiviral efficacy for at least one week at room temperature (e.g., about 20-40° C.). In certain embodiments, the composition retains antiviral efficacy for at least two weeks at room temperature (e.g., about 20-40° C.). In certain embodiments, the composition retains antiviral efficacy for at least three weeks at room temperature (e.g., about 20-40° C.). In certain embodiments, the composition retains antiviral efficacy for at least 1 month at room temperature (e.g., about 20-40° C.). In certain embodiments, the composition retains antiviral efficacy for at least 2 months at room temperature (e.g., about 20-40° C.). In certain embodiments, the composition retains antiviral efficacy for at least 3 months at room temperature (e.g., about 20-40° C.). In certain embodiments, the composition retains antiviral efficacy for at least 4 months at room temperature (e.g., about 20-40° C.). In certain embodiments, the composition retains antiviral efficacy for at least 5 months at room temperature (e.g., about 20-40° C.). In certain embodiments, the composition retains antiviral efficacy for at least 6 months at room temperature (e.g., about 20-40° C.). In certain embodiments, the composition retains antiviral efficacy for at least 7 months at room temperature (e.g., about 20-40° C.). In certain embodiments, the composition retains antiviral efficacy for at least 8 months at room temperature (e.g., about 20-40° C.). In certain embodiments, the composition retains antiviral efficacy for at least 9 months at room temperature (e.g., about 20-40° C.). In certain embodiments, the composition retains antiviral efficacy for at least 10 months at room temperature (e.g., about 20-40° C.). In certain embodiments, the composition retains antiviral efficacy for at least 11 months at room temperature (e.g., about 20-40° C.). In certain embodiments, the composition retains antiviral efficacy for at least 1 year at room temperature (e.g., about 20-40° C.). In certain embodiments, the composition retains antiviral efficacy for at least 2 years at room temperature (e.g., about 20-40° C.). In certain embodiments, the composition retains antiviral efficacy for at least 3 years at room temperature (e.g., about 20-40° C.). In certain embodiments, the composition retains antiviral efficacy for at least 5 years at room temperature (e.g., about 20-40° C.). In certain embodiments, the composition retains antiviral efficacy for at least 10 years at room temperature (e.g., about 20-40° C.).

Methods of Treatment and/or Prevention and Uses

The present disclosure provides methods of treating a viral infection (e.g., infection by coronavirus, ebolavirus, Lassa virus) in a subject, comprising intranasal administration of a composition described herein, which may be optionally administered in combination with an additional pharmaceutical agent. The present disclosure also provides methods of preventing a viral infection (e.g., infection by a coronavirus, an ebolavirus, a Lassa virus) in a subject, comprising intranasal administration of a composition described herein, which may be optionally administered in combination with an additional pharmaceutical agent. The present disclosure also provides methods of treating damage to respiratory tissue (e.g. upper respiratory tissue, for example, nasal tissue, nasopharyngeal tissue, and/or lower respiratory tissue, for example, lung tissue) in a subject, comprising intranasal administration of a composition described herein, which may be optionally administered in combination with an additional pharmaceutical agent. The present disclosure also provides methods of treating damage to respiratory tissue (e.g. upper respiratory tissue, for example, nasal tissue, nasopharyngeal tissue, and/or lower respiratory tissue, for example, lung tissue) in a subject, comprising intranasal administration of a composition described herein, which may be optionally administered in combination with an additional pharmaceutical agent. The present disclosure also provides methods of preventing damage to respiratory tissue (e.g. upper respiratory tissue, for example, nasal tissue, nasopharyngeal tissue, and/or lower respiratory tissue, for example, lung tissue) in a subject, comprising intranasal administration of a composition described herein, which may be optionally administered in combination with an additional pharmaceutical agent. The present disclosure also provides the compositions described herein to treat and/or prevent a viral infection (e.g., infection by a coronavirus, an ebolavirus, a Lassa virus) caused by a virus in a subject in need thereof.

In another aspect, the present disclosure provides methods of treating a viral infection (e.g., infection by coronavirus, ebolavirus, Lassa virus) in a subject, comprising intranasal administration of a composition described herein, which may be optionally administered in combination with an additional pharmaceutical agent. In certain embodiments, the virus is a coronavirus, an ebolavirus, or a Lassa virus. In certain embodiments, the virus is severe acute respiratory syndrome coronavirus 1 (SARS-CoV-1). In certain embodiments, the virus is severe acute respiratory syndrome coronavirus 2 (SARS-CoV-2). In certain embodiments, the virus is Middle East respiratory syndrome-related coronavirus (MERS-CoV). In certain embodiments, the virus causes coronavirus disease 2019 (COVID-19).

In another aspect, the present disclosure provides methods of preventing a viral infection (e.g., infection by coronavirus, ebolavirus, Lassa virus) in a subject, comprising intranasal administration of a composition described herein, which may be optionally administered in combination with an additional pharmaceutical agent. In certain embodiments, the virus is a coronavirus, an ebolavirus, or a Lassa virus. In certain embodiments, the virus is severe acute respiratory syndrome coronavirus 1 (SARS-CoV-1). In certain embodiments, the virus is severe acute respiratory syndrome coronavirus 2 (SARS-CoV-2). In certain embodiments, the virus is Middle East respiratory syndrome-related coronavirus (MERS-CoV). In certain embodiments, the virus causes coronavirus disease 2019 (COVID-19).

In another aspect, the present disclosure provides methods of treating damage to respiratory tissue (e.g. upper respiratory tissue, for example, nasal tissue, nasopharyngeal tissue, and/or lower respiratory tissue, for example, lung tissue) in a subject, comprising intranasal administration of a composition described herein, which may be optionally administered in combination with an additional pharmaceutical agent. In certain embodiments, the respiratory tissue is upper respiratory tissue (e.g. nasal tissue, nasopharyngeal tissue). In certain embodiments, the upper respiratory tissue is nasal tissue or nasopharyngeal tissue. In certain embodiments, the respiratory tissue is lower respiratory tissue (e.g. lung tissue). In certain embodiments, the lower respiratory tissue is lung tissue.

In another aspect, the present disclosure provides methods of preventing damage to respiratory tissue (e.g. upper respiratory tissue, for example, nasal tissue, nasopharyngeal tissue, and/or lower respiratory tissue, for example, lung tissue) in a subject, comprising intranasal administration of a composition described herein, which may be optionally administered in combination with an additional pharmaceutical agent. In certain embodiments, the respiratory tissue is upper respiratory tissue (e.g. nasal tissue, nasopharyngeal tissue). In certain embodiments, the upper respiratory tissue is nasal tissue or nasopharyngeal tissue. In certain embodiments, the respiratory tissue is lower respiratory tissue (e.g. lung tissue). In certain embodiments, the lower respiratory tissue is lung tissue.

In certain embodiments, the intranasal administration to the subject is before exposure of the subject to the virus. In certain embodiments, the intranasal administration to the subject is immediately before exposure of the subject to the virus. In certain embodiments, the intranasal administration to the subject is within 1 hour before exposure of the subject to the virus. In certain embodiments, the intranasal administration to the subject is within 3 hours before exposure of the subject to the virus. In certain embodiments, the intranasal administration to the subject is within 6 hours before exposure of the subject to the virus. In certain embodiments, the intranasal administration to the subject is within 12 hours before exposure of the subject to the virus. In certain embodiments, the intranasal administration to the subject is within 24 hours before exposure of the subject to the virus. In certain embodiments, the intranasal administration to the subject is within 48 hours before exposure of the subject to the virus. In certain embodiments, the intranasal administration to the subject is within 72 hours before exposure of the subject to the virus. In certain embodiments, the intranasal administration to the subject is within 96 hours before exposure of the subject to the virus.

In certain embodiments, the intranasal administration to the subject is after exposure of the subject to the virus. In certain embodiments, the intranasal administration to the subject is immediately after exposure of the subject to the virus. In certain embodiments, the intranasal administration to the subject is within 1 hour of exposure of the subject to the virus. In certain embodiments, the intranasal administration to the subject is within 3 hours of exposure of the subject to the virus. In certain embodiments, the intranasal administration to the subject is within 6 hours of exposure of the subject to the virus. In certain embodiments, the intranasal administration to the subject is within 12 hours of exposure of the subject to the virus. In certain embodiments, the intranasal administration to the subject is within 24 hours of exposure of the subject to the virus. In certain embodiments, the intranasal administration to the subject is within 48 hours of exposure of the subject to the virus. In certain embodiments, the intranasal administration to the subject is within 72 hours of exposure of the subject to the virus. In certain embodiments, the intranasal administration to the subject is within 96 hours of exposure of the subject to the virus.

In certain embodiments, the intranasal administration is repeated one or more additional times. In certain embodiments, the intranasal administration is repeated two or more additional times. In certain embodiments, the intranasal administration is repeated three or more additional times. In certain embodiments, the intranasal administration is repeated four or more additional times. In certain embodiments, the intranasal administration is repeated five or more additional times. In certain embodiments, the intranasal administration is repeated six or more additional times. In certain embodiments, the intranasal administration is repeated seven or more additional times. In certain embodiments, the intranasal administration is repeated eight or more additional times. In certain embodiments, the intranasal administration is repeated nine or more additional times. In certain embodiments, the intranasal administration is repeated ten or more additional times.

In certain embodiments, the intranasal administration is via nasal drops. In certain embodiments, the intranasal administration is via a nasal spray. In certain embodiments, the intranasal administration is via inhalation, nebulization, or aerosolization. In certain embodiments, the intranasal administration comprises inhaling through the nose. In certain embodiments, the intranasal administration comprises inhaling through the mouth. In certain embodiments, the intranasal administration comprises inhaling through the nose and mouth.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal, such as a transgenic mouse or transgenic pig.

In certain embodiments, the methods comprising intranasal administration of a composition described herein are useful for inhibiting the activity of a protein kinase (e.g. PIKfyve kinase) in a subject or biological sample (e.g., tissue, cell). In certain embodiments, the methods comprising intranasal administration of a composition described herein are useful for inhibiting the activity of a serine protease (e.g. TMPRSS2) in a subject or biological sample (e.g., tissue, cell). In certain embodiments, the methods comprising intranasal administration of a composition described herein are useful for inhibiting the activity of a serine protease (e.g. TMPRSS2) in a subject or biological sample (e.g., tissue, cell), and treating and/or preventing a viral infection (e.g., infection by coronavirus, ebolavirus, Lassa virus).

In another aspect, the present disclosure provides the compositions described herein to treat and/or prevent a viral infection (e.g., infection by coronavirus, ebolavirus, Lassa virus) caused by a virus in a subject in need thereof.

In another aspect, the present disclosure provides the compositions described herein to treat and/or prevent damage to respiratory tissue (e.g. upper respiratory tissue, for example, nasal tissue, nasopharyngeal tissue, and/or lower respiratory tissue, for example, lung tissue) in a subject in need thereof.

In another aspect, the present disclosure provides the compositions described herein for use in a method described herein (e.g., a method of treating and/or preventing a viral infection (e.g., infection by a coronavirus, an ebolavirus, a Lassa virus) caused by a virus in a subject in need thereof, or a method of treating and/or preventing damage to respiratory tissue (e.g. upper respiratory tissue, for example, nasal tissue, nasopharyngeal tissue, and/or lower respiratory tissue, for example, lung tissue) in a subject in need thereof).

In another aspect, the present disclosure provides the compositions described herein for use in treating and/or preventing a viral infection (e.g., infection by a coronavirus, an ebolavirus, a Lassa virus) caused by a virus in a subject in need thereof. In certain embodiments, the virus is severe acute respiratory syndrome coronavirus 1 (SARS-CoV-1). In certain embodiments, the virus is severe acute respiratory syndrome coronavirus 2 (SARS-CoV-2). In certain embodiments, the virus is Middle East respiratory syndrome-related coronavirus (MERS-CoV). In certain embodiments, the virus causes coronavirus disease 2019 (COVID-19).

In another aspect, the present disclosure provides the compositions described herein for use in treating and/or preventing damage to respiratory tissue (e.g. upper respiratory tissue, for example, nasal tissue, nasopharyngeal tissue, and/or lower respiratory tissue, for example, lung tissue) in a subject in need thereof. In certain embodiments, the respiratory tissue is upper respiratory tissue (e.g. nasal tissue, nasopharyngeal tissue). In certain embodiments, the upper respiratory tissue is nasal tissue or nasopharyngeal tissue. In certain embodiments, the respiratory tissue is lower respiratory tissue (e.g. lung tissue). In certain embodiments, the lower respiratory tissue is lung tissue.

In another aspect, the present disclosure provides the compositions described herein for use in the manufacture of a medicament for the treatment and/or prevention of a viral infection (e.g., infection by a coronavirus, an ebolavirus, a Lassa virus) caused by a virus in a subject in need thereof. In certain embodiments, the virus is severe acute respiratory syndrome coronavirus 1 (SARS-CoV-1). In certain embodiments, the virus is severe acute respiratory syndrome coronavirus 2 (SARS-CoV-2). In certain embodiments, the virus is Middle East respiratory syndrome-related coronavirus (MERS-CoV). In certain embodiments, the virus causes coronavirus disease 2019 (COVID-19).

In another aspect, the present disclosure provides the compositions described herein for use in the manufacture of a medicament for the treatment and/or prevention of damage to respiratory tissue (e.g. upper respiratory tissue, for example, nasal tissue, nasopharyngeal tissue, and/or lower respiratory tissue, for example, lung tissue) in a subject in need thereof. In certain embodiments, the respiratory tissue is upper respiratory tissue (e.g. nasal tissue, nasopharyngeal tissue). In certain embodiments, the upper respiratory tissue is nasal tissue or nasopharyngeal tissue. In certain embodiments, the respiratory tissue is lower respiratory tissue (e.g. lung tissue), In certain embodiments, the lower respiratory tissue is lung tissue.

### Kits

Also encompassed by the disclosure are kits (e.g., pharmaceutical packs). The kits provided may comprise a pharmaceutical composition described herein and a container (e.g., a vial, ampule, bottle, syringe, nasal spray applicator, nasal spray pump, and/or dispenser package, or other suitable container). In some embodiments, provided kits may optionally further include a second container comprising a pharmaceutical excipient for dilution or suspension of a pharmaceutical composition described herein. In some embodiments, the pharmaceutical excipient is a preservative. In some embodiments, the pharmaceutical composition described herein provided in the first container and the second container are combined to form one unit dosage form. In some embodiments, provided kits may optionally comprise a plurality of single dose containers (e.g., vials, ampules, bottles, syringes, nasal spray applicators, nasal spray pumps, and/or dispenser packages, or other suitable containers).

Thus, in one aspect, provided are kits including a first container comprising a pharmaceutical composition described herein. In certain embodiments, the kits are useful for treating a disease (e.g., viral infection) in a subject in need thereof. In certain embodiments, the kits are useful for preventing a disease (e.g., viral infection, for example, infection by coronavirus, ebolavirus, Lassa virus) in a subject in need thereof. In certain embodiments, the kits are useful for reducing the risk of developing a disease (e.g., viral infection, for example, infection by coronavirus, ebolavirus, Lassa virus) in a subject in need thereof. In certain embodiments, the kits are useful for inhibiting the activity of a protein kinase (e.g. PIKfyve kinase) in a subject or biological sample (e.g., tissue, cell). In certain embodiments, the kits are useful for inhibiting the activity of a serine protease (e.g. TMPRSS2) in a subject or biological sample (e.g., tissue, cell).

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., viral infection) in a subject in need thereof. In certain embodiments, the kits and instructions provide for preventing a disease (e.g., viral infection) in a subject in need thereof. In certain embodiments, the kits and instructions provide for reducing the risk of developing a disease (e.g., viral infection) in a subject in need thereof. In certain embodiments, the kits and instructions provide for inhibiting the activity of a protein kinase (e.g. PIKfyve kinase) in a subject or biological sample (e.g., tissue, cell). In certain embodiments, the kits and instructions provide for inhibiting the activity of a serine protease (e.g. TMPRSS2) in a subject or biological sample (e.g., tissue, cell). A kit described herein may include one or more additional pharmaceutical agents described herein as a separate composition.

### EXAMPLES

### Materials and Methods

Animals. 8 week old, female, BALB C mice were used.

Virus. SARS-CoV-2 beta variant (i.e., South African variant) was used. Human isolate of Virus SARS-CoV-2 beta from an individual was inoculated into Vero E6-TMPRSS2 cells grown in MEM 2% FBS, and medium containing virus collected two days post infection. The supernatant was centrifuged at 3000 g for 10 minutes to remove cell debris and the supernatant aliquoted and stored at −80° C. Virus genome of isolate before and after growth in culture was verified by deep sequencing and confirmed to be the same.

Infection dose. 5×10⁵ pfu/mouse in 20 ul of MEM medium plus 2% FCS (FCS-heat inactivated foctal bovine serum). The virus is a supernatant from VERO-TMPRSS2 cells as described above.

Virus delivery protocol. The animals were anesthetized, placed in horizontal position belly up, the head tilted backward (the animal head is held from the back of the neck) so that the nose is pointing up. 50 ul of virus suspension was slowly delivered in front of the animal nose. The animal inhales the liquid through both nostrils [DOI:10.3390/v13112263, Kant, R. et al. Common Laboratory Mice Are Susceptible to Infection with the SARS-CoV-2 Beta Variant. Viruses 13, 2263 (2021)].

Drug delivery protocol. The animals were anesthetized, 50 ul of aqueous solution containing the drug mixture was slowly delivered into the front of the same nostril used for virus delivery while the animal was belly up and breathed normally. Drugs delivered intranasally about the same time of infection or 6 hours post infection. Drugs further delivered 12, 24 and 36 hr post infection [DOI:10.3390/v13112263, Kant, R. et al. Common Laboratory Mice Are Susceptible to Infection with the SARS-CoV-2 Beta Variant. Viruses 13, 2263 (2021)].

Drug solutions. Solutions of Apilimod dimesylate (25 mg/ml) and Nafamostat mesylate (10 mg/ml) were made (by simple suspension) in deionized water (MilliQ) and stored at either room temperature (˜23.5° C.) or frozen (−20° C.).

Solutions at different final concentrations of Apilimod dimesylate and Nafamostat mesylate alone or as a mixture were made using the stock solutions added to deionized (MilliQ) water. The drugs (alone or as a mixture) were delivered intranasally either 20-30 second before or 6 hours after intranasal administration of the virus. Complete stability of the drug mixtures made in deionized (MilliQ) water and kept at 23.5° C. for one month was verified by showing the same antiviral efficacy as an aliquot solution kept frozen when tested 24 h after infection of Caco2-ACE2 cells. Complete stability of the drug mixtures made in deionized (MilliQ) water and kept at 23.5° C. for three months was verified by showing the same antiviral efficacy as an aliquot solution kept frozen when tested in VeroE6-TMPRSS2 cell cultures.

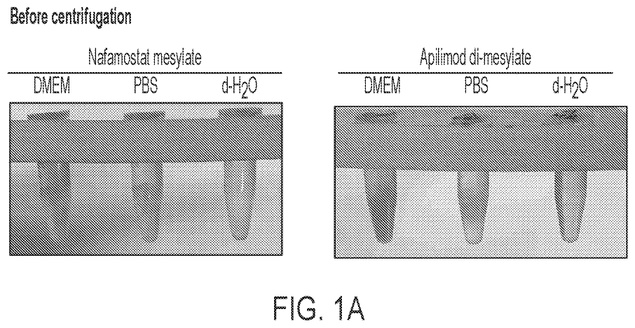

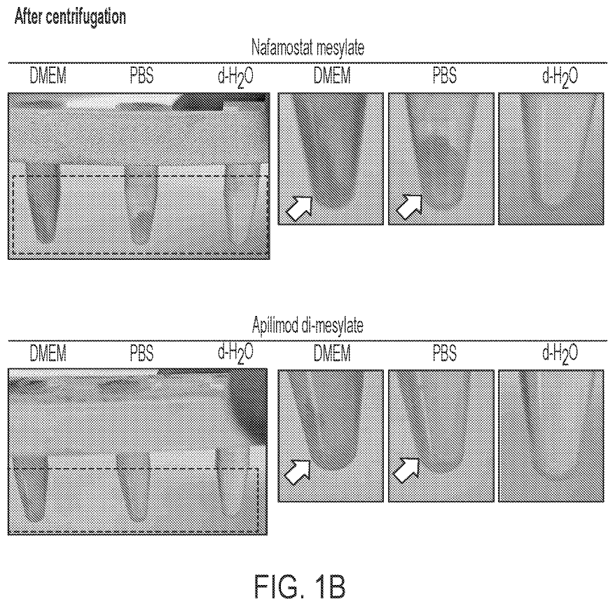

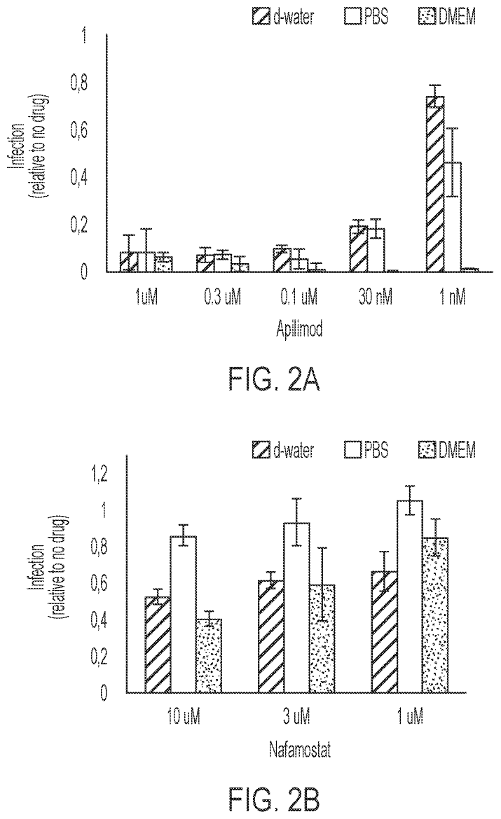

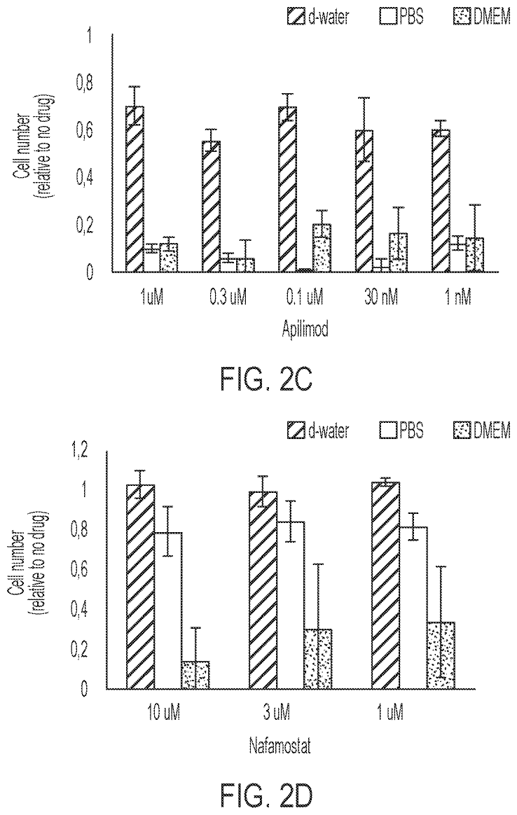

Apilimod dimesylate and Nafamostat mesylate, particularly when together, are relatively insoluble and precipitate in solutions containing salt such as phosphate buffer saline (PBS) or DMEM cell culture medium. It was empirically uncovered that solubility of these compounds substantially increased when mixed and dissolved in deionized (MilliQ) water (FIGS. 1A-1B). This is the reason why our drug mixtures were made in deionized (MilliQ) water as stocks and for intranasal administration. While the mouse nose tolerates exposure to deionized (MilliQ) water, for humans it may be necessary to adjust the osmolarity of the solution, for example by adding sucrose.

Animal infection tests. Two days post infection the mice were anesthetized according to an approved protocol and appropriate organs retrieved two days after infection for RNA extraction and PCR testing and for histopathology assessment using chemically fixed tissues embedded in paraffin.

The PCR reaction was carried using three different set of primers, whose sequences were specific for the viral protein E, RdRNP, and sub-genomic-E, respectively. The results obtained with each set of primers were similar and included comparable PCR-value fluctuations consistent with variations in pipetting errors and lack of isogeneity amongst the mice, Pathology-based apoptosis assay is based on immune staining of lung sections using an antibody against cleaved caspase 3.

All animal infection and animal infection tests were conducted as blind experiments,

### Example 1: Full Infection Block by Intranasal Delivery of a Mixture of Apilimod Dimesylate and Nafamostat Mesylate

Simultaneous intranasal delivery of a mixture of Apilimod dimesylate and Nafamostat mesylate briefly preceding by ˜30 seconds the intranasal delivery of virus into the same nostril. Complete block of infection as determined two days post infection by undetectable PCR signal in the lung: one third of the right lung was used for PCR; left lung was used for histopathology analysis [DOI:10.3390/v13112263, Kant, R. et al. Common Laboratory Mice Are Susceptible to Infection with the SARS-CoV-2 Beta Variant. Viruses 13, 2263 (2021)]. Histopathological evaluation of the lungs showed a healthy tissue, with no indication of apoptosis or signs of immune cell infiltration.

Complete block of infection by detectable PCR signal in the lung two days post infection after using mixtures with different combinations of drug amounts: complete infection block observed following combined intranasal delivery of 25 μL deionized water containing 0.8 or 0.4 mg Apilimod dimesylate/Kg mice together with 0.2 mg Nafamostat mesylate/Kg mice (a typical mouse weight is 20 gr).

### Example 2: Partial or No Block of Infection by Intranasal Delivery of Apilimod Dimesylate or Nafamostat Mesylate Alone

Intranasal delivery of Apilimod dimesylate or Nafamostat mesylate briefly preceding by ˜30 seconds the intranasal delivery of virus into the same nostril. Infection tests carried two days post infection.

Apilimod dimesylate had no antiviral effect at 2 mg/Kg.

Nafamostat mesylate at 2 mg/Kg had no effect and at 4 mg/Kg reduced infection (PCR) by 5-10-fold.

### Example 3: Positive Infection Control

Mice were exposed to virus but no drugs as positive controls and were then similarly evaluated two days post infection. These animals became infected based on a positive PCR signal (viral genes E, subgenomic-E, and RNA-dependent RNA polymerase) in the lung and prominent immune staining of cells labelled with antibodies specific for viral protein S. Their lungs appeared unhealthy as determined by extensive apoptosis (by immune staining with antibody specific for cleaved-caspase-3) and modest infiltration of immune cells in the infected areas.

### Example 4: Animal Test Where the Drug Combination is Given 0 (e.g., 30 Sec Prior Infection), 6,24 and 30 Hours Post Infection and Block of Infection Tested 2 Days Post Infection

The animals in the positive control experiments (non-drug treated) became infected based on a positive PCR signal (viral genes E, subgenomic-E, and RNA-dependent RNA polymerase) in the lung and immune staining of cells labelled with antibodies specific for viral protein S. Unlike the non-drug treated infected positive controls, the drug treated animals displayed complete absence or very low levels of tissue damage in their lungs, as determined by histopathology evaluation including minimal apoptosis (by immune staining with antibody specific for cleaved-caspase-3) and minimal infiltration of immune cells in the infected areas.

This experiment confirms that the drug combination prevents infection and strongly reduces lung tissue damage when administered during and after infection.

### Example 5: Animal Test in Which Drug Combination is Given 0 (e.g. 30 Sec Prior Infection), 6, 24 and 30 Hours Post Infection and Block of Infection Tested 4 Days Post Infection

This experiment tests whether drug treatment fully eradicated the infection or whether the residual viruses are sufficient to re-initiate infection and cause disease two days after termination of drug treatment.

Unlike the non-drug treated infected positive controls in Example 3, these animals displayed no infection based on a negative PCR signal (viral genes E, subgenomic-E, and RNA-dependent RNA polymerase) in the lung and negative immune staining of cells labelled with antibodies specific for viral protein S. These drugs treated animals also showed complete absence or very low levels of tissue damage in their lungs, as determined by histopathology evaluation including no or minimal apoptosis (by immune staining with antibody specific for cleaved-caspase-3) and minimal infiltration of immune cells in the infected areas.

This experiment confirms that the drug combination prevents infection and strongly reduces lung tissue damage when administered during and after infection. It also shows that two days after drug treatment, potential residual viruses are not sufficient to re-initiate infection in the lungs when assayed four days after infection.

### Example 6: Animal Test Where the Drug Combination is Given 6, 12, 24, and 30 Hours Post Infection, and Block of Infection Tested 2 Days Post Infection

This experiment tests whether the drug combination has positive therapeutic effect even when administered after infection.

These animals became infected based on a positive PCR signal (viral genes E, subgenomic-E, and RNA-dependent RNA polymerase) in the lung and immune staining of cells labelled with antibodies specific for viral protein S. Unlike the non-drug treated infected positive controls in Example 3, these animals displayed complete absence or very low levels of tissue damage in their lungs, as determined by histopathology evaluation and minimal apoptosis (by immune staining with antibody specific for cleaved-caspase-3) in the infected areas.

This experiment confirms that the drug combination prevents or strongly reduces lung tissue damage even if administered after infection.

### Incorporation by Reference

The present application refers to various issued patent, published patent applications, scientific journal articles, and other publications, all of which are incorporated herein by reference. The details of one or more embodiments of the disclosure are set forth herein. Other features, objects, and advantages of the disclosure will be apparent from the Detailed Description, the Figures, the Examples, and the Claims.

### Equivalents and Scope

In the claims and throughout, articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim, Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features, For purposes of simplicity, those embodiments have not been specifically set forth in haec verba herein. It is also noted that the terms “comprising” and “containing” are intended to be open and permits the inclusion of additional elements or steps, Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the disclosure can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present disclosure, as defined in the following claims.

What is claimed is:

1. A composition comprising:
(a) a phosphatidylinositol-3-phosphate 5-kinase (PIKfyve kinase) inhibitor, or a pharmaceutically acceptable salt, solvate, hydrate, stereoisomer, polymorph, tautomer, isotopically enriched form, or prodrug thereof;
(b) a serine protease inhibitor, or a pharmaceutically acceptable salt, solvate, hydrate, stereoisomer, polymorph, tautomer, isotopically enriched form, or prodrug thereof, and
(c) water.

2. The composition of claim 1, wherein the serine protease inhibitor inhibits transmembrane serine protease 2 (TMPRSS2).

3. The composition of any one of claim 1 or 2, wherein the PIKfyve kinase inhibitor is apilimod or vacuolin-1, or a pharmaceutically acceptable salt thereof.

4. The composition of any one of claims 1-3, wherein the PIKfyve kinase inhibitor is apilimod, or a pharmaceutically acceptable salt thereof.

5. The composition of any one of claims 1-4, wherein the PIKfyve kinase inhibitor is apilimod dimesylate.

6. The composition of any one of claims 1-5, comprising about 1-50 mg/mL apilimod dimesylate.

7. The composition of any one of claims 1-6, comprising about 10-30 mg/mL apilimod dimesylate.

8. The composition of any one of claims 1-7, comprising about 15-20 mg/mL apilimod dimesylate.

9. The composition of any one of claims 1-8, comprising about 18 mg/mL apilimod dimesylate.

10. The composition of any one of claims 1-9, wherein the serine protease inhibitor is nafamostat or camostat, or a pharmaceutically acceptable salt thereof.

11. The composition of any one of claims 1-10, wherein the serine protease inhibitor is nafamostat mesylate.

12. The composition of any one of claims 1-11, comprising about 1-100 nafamostat mesylate.

13. The composition of any one of claims 1-12, comprising about 10-50 mg/mL nafamostat mesylate.

14. The composition of any one of claims 1-13, comprising about 30-40 mg/mL nafamostat mesylate.

15. The composition of any one of claims 1-14, comprising about 36 mg/mL nafamostat mesylate.

16. The composition of any one of claim 1-6 or 10-12, comprising about 1-50 mg/mL apilimod dimesylate and about 1-100 mg/mL nafamostat mesylate.

17. The composition of any one of claim 1-7 or 10-13, comprising about 10-30 mg/mL apilimod dimesylate and about 10-50 mg/mL nafamostat mesylate.

18. The composition of any one of claim 1-8 or 10-14, comprising about 15-20 mg/mL apilimod dimesylate and about 30-40 mg/mL nafamostat mesylate.

19. The composition of any one of claim 1-9 or 10-15, comprising about 18 mg/mL apilimod dimesylate and about 36 mg/mL nafamostat mesylate.

20. The composition of any one of claims 1-19, further comprising sucrose.

21. The composition of any one of claims 1-20, wherein the composition comprises a solution of:
(a) a phosphatidylinositol-3-phosphate 5-kinase (PIKfyve kinase) inhibitor, or a pharmaceutically acceptable salt, solvate, hydrate, stereoisomer, polymorph, tautomer, isotopically enriched form, or prodrug thereof;
(b) a serine protease inhibitor, or a pharmaceutically acceptable salt, solvate, hydrate, stereoisomer, polymorph, tautomer, isotopically enriched form, or prodrug thereof, and
(c) water.

22. The composition of any one of claims 1-21, wherein the water is deionized water.

23. The composition of any one of claims 1-22, wherein the water is distilled water.

24. The composition of any one of claims 1-23, wherein the composition has a pH of about 2.0-12.0.

25. The composition of any one of claims 1-24, wherein the composition has a pH of about 2.0-8.0.

26. The composition of any one of claims 1-25, wherein the composition has a pH of about 2.0-5.0.

27. The composition of any one of claims 1-26, wherein the composition has a pH of about 3.0-4.5.

28. The composition of any one of claims 1-27, wherein the deionized water has a salt concentration of less than about 150 mM.

29. The composition of any one of claims 1-28, further comprising an additional solvent.

30. The composition of claim 29, wherein the additional solvent is a polar aprotic solvent.

31. The composition of claim 30, wherein the polar aprotic solvent is DMSO.

32. The composition of claim 29, wherein the additional solvent is an alcohol.

33. The composition of claim 32, wherein the alcohol is ethanol.

34. The composition of any one of claims 1-33, wherein the composition is stable for at least two weeks.

35. The composition of any one of claims 1-34, wherein the composition is stable for at least two weeks at room temperature.

36. The composition of any one of claims 1-35, wherein the composition is stable for at least 1 month.

37. The composition of any one of claims 1-36, wherein the composition is stable for at least 3 months.

38. The composition of any one of claims 1-37, wherein the composition is stable for at least 6 months.

39. The composition of any one of claims 1-38, wherein the composition is stable at about 0-40° C.

40. The composition of any one of claims 1-39, wherein the composition is stable at about 4° C.

41. The composition of any one of claims 1-39, wherein the composition is stable at about 20-40° C.

42. The composition of any one of claim 1-39 or 41, wherein the composition is stable at about 23.5° C.

43. The composition of any one of claims 1-39, wherein the composition is stable at about 37° C.

44. The composition of any one of claims 1-43, wherein the composition retains antiviral efficacy.

45. The composition of any one of claims 1-44, comprising apilimod dimesylate and nafamostat mesylate.

46. The composition of any one of claims 1-45, comprising about 1 mg/mL apilimod dimesylate and about 2 mg/mL nafamostat mesylate.

47. A method of treating and/or preventing a viral infection in a subject, the method comprising intranasal administration of a composition of any one of claims 1-46 to the subject.

48. The method of claim 47, wherein the virus is a coronavirus, an ebolavirus, or a Lassa virus.

49. The method of any one of claim 47 or 48, wherein the virus is severe acute respiratory syndrome coronavirus 1 (SARS-CoV-1).

50. The method of any one of claim 47 or 48, wherein the virus is severe acute respiratory syndrome coronavirus 2 (SARS-CoV-2).

51. The method of any one of claim 47 or 48, wherein the virus is Middle East respiratory syndrome-related coronavirus (MERS-CoV).

52. The method of any one of claim 47, 48, or 50, wherein the virus causes coronavirus disease 2019 (COVID-19).

53. A method of treating and/or preventing damage to respiratory tissue in a subject, the method comprising intranasal administration of a composition of any one of claims 1-46 to the subject.

54. The method of claim 53, wherein the respiratory tissue is upper respiratory tissue.

55. The method of claim 54, wherein the upper respiratory tissue is nasal tissue or nasopharyngeal tissue.

56. The method of claim 53, wherein the respiratory tissue is lower respiratory tissue.

57. The method of claim 56, wherein the lower respiratory tissue is lung tissue.

58. The method of any one of claims 47-57, wherein the intranasal administration to the subject is before exposure of the subject to the virus.

59. The method of any one of claims 47-57, wherein the intranasal administration to the subject is after exposure of the subject to the virus.

60. The method of any one of claims 47-59, wherein the intranasal administration is repeated one or more additional times.

61. The method of any one of claims 47-60, wherein the intranasal administration is repeated at least four additional times.

62. The method of any one of claims 47-61, wherein the intranasal administration is via nasal drops.

63. The method of any one of claims 47-61, wherein the intranasal administration is via a nasal spray.

64. The method of any one of claims 47-61, wherein the intranasal administration is via inhalation, nebulization, or aerosolization.

65. Use of a composition of any one of claims 1-46 to treat and/or prevent a viral infection caused by a virus in a subject in need thereof.

66. Use of a composition of any one of claims 1-46 to treat and/or prevent damage to respiratory tissue in a subject in need thereof.

67. A kit comprising:
(a) the composition of any one of claims 1-46; and
(b) instructions for use of the composition.
